(19) United States
(12) Patent Application Publication
(10) Pub. No.: US 2025/0188150 A1
(43) Pub. Date: Jun. 12, 2025

(54) PEPTIDES FOR THE TREATMENT OF FIBROSIS

(71) Applicant: Technion Research & Development Foundation Limited, Haifa (IL)

(72) Inventors: Peleg HASSON, Haifa (IL); Haguy WOLFENSON, Haifa (IL); Shay MELAMED, Haifa (IL); Shelly EILOT, Haifa (IL); Elisabeth BOGER, Haifa (IL); Rohtem AVIRAM, Haifa (IL)

(73) Assignee: Technion Research & Development Foundation Limited, Haifa (IL)

(21) Appl. No.: 19/050,148

(22) Filed: Feb. 11, 2025

Related U.S. Application Data
(63) Continuation of application No. PCT/IL2023/050840, filed on Aug. 10, 2023.
(60) Provisional application No. 63/396,968, filed on Aug. 11, 2022.

(51) Int. Cl. C07K 14/78; A61K 38/00; A61P 35/00; A61P 43/00

(52) U.S. Cl. CPC C07K 14/78; A61P 35/00; A61P 43/00; A61K 38/00; C07K 2319/30

(57) ABSTRACT

An isolated peptide comprising an amino acid sequence of a lysyl oxidase (LOX) family member target site of an extracellular matrix protein, the peptide being no longer than 50 amino acids, for use in treating a disease associated with fibrosis in a subject in need thereof.

### RELATED APPLICATIONS

This application is a Continuation of PCT Patent Application No. PCT/1L2023/050840 having International filing date of Aug. 10, 2023, which claims the benefit of priority under 35 USC § 119(e) of U.S. Provisional Patent Application No. 63/396,968 filed on Aug. 11, 2022. The contents of the above applications are all incorporated by reference as if fully set forth herein in their entirety.

### SEQUENCE LISTING STATEMENT

The XML file, entitled 102356SequenceListing.xml, created on Dec. 8, 2024, comprising 61,459 bytes, submitted concurrently with the filing of this application is incorporated herein by reference. The sequence listing submitted herewith is identical to the sequence listing forming part of the international application.

### FIELD AND BACKGROUND OF THE INVENTION

The present invention, in some embodiments thereof, relates to peptides for the treatment of diseases associated with fibrosis.

Fibrosis is the abnormal accumulation of fibrous extracellular matrix, mostly observed as a response to injury or inflammation. Approximately 45% of mortality in the Western world is attributed to a group of conditions known as fibrotic diseases. Altogether, these diseases are more frequent than cancer or vascular diseases. These fibrotic conditions include systemic diseases such as cystic fibrosis, systemic sclerosis, and scleroderma, where multiple organs are affected, or ones in which an individual organ is affected, such as the lung, liver, kidneys, skeletal muscles, skin, or heart. Further, while fibrosis can be the primary condition or a secondary response (e.g., cardiotoxicity following chemotherapy, post-operative fibrosis, diabetic fibrosis etc.), it also promotes primary tumor growth and metastatic spread. Thus, regardless of the cause, fibrosis acts as a major driver of the pathological condition. As a result, fibrosis inhibition is at the heart of multiple clinical trials (over 1000), yet thus far with poor success.

Although fibrosis is caused by a variety of distinct diseases and genetic mutations, some basic underlying molecular processes are shared between them all. Two core fibrotic proteins are the extracellular matrix fibrillar protein Fibronectin and members of the Lysyl oxidase enzyme family that are upregulated and involved in all fibrotic processes. Indeed, inhibition of lysyl oxidases' activity has been shown to reduce fibrosis, and lack of fibronectin fibrillogenesis prevents fibrosis. Still, no therapeutic agent is available to target either one.

Current treatments mainly involve the use of inhibitors of the TGFβ pathway, which is involved in extracellular matrix secretion, or steroids to reduce the inflammatory response that is involved in fibrosis. The TGFβ pathway is an essential regulator of the most fundamental cell and tissue functions, and therefore the use of TGFβ inhibitors leads to multiple adverse effects, including blurred vision, difficulty breathing, and irregular heart bit, among others, and therefore prevents their chronic use. Moreover, TGFβ inhibition does not demonstrate a significant improvement in all fibrotic diseases, such as Duchenne Muscular Dystrophy. The chronic use of steroids also has multiple adverse effects, including increased appetite, weight gain, changes in mood, muscle weakness, blurred vision, and low resistance to infections. Therefore, many patients prefer not to be treated with steroids for long periods.

Background art includes Fogelgren et al., J Biol Chem. 2005 Jul. 1; 280(26):24690-7. doi: 10.1074/jbc.M412979200. Epub 2005 Apr. 19. PMID: 15843371 which discloses that fibroncectin binds to lysyl oxidase (LOX).

### SUMMARY OF THE INVENTION

According to an aspect of the present invention there is provided an isolated peptide comprising an amino acid sequence of a lysyl oxidase (LOX) family member target site of an extracellular matrix protein, the peptide being no longer than 50 amino acids, for use in treating a disease associated with fibrosis in a subject in need thereof.

According to an embodiment of the present invention, the peptide comprises a modification which imparts the peptide with enhanced stability under physiological conditions as compared to a native form of the peptide not comprising the modification.

According to an embodiment of the present invention, the modification comprises a proteinaceous modification.

According to an embodiment of the present invention, the proteinaceous modification is selected from the group consisting of immunoglobulin, human serum albumin, and transferrin.

According to an embodiment of the present invention, the immunoglobulin comprises an Fc domain.

According to an embodiment of the present invention, the modification comprises a chemical modification.

According to an embodiment of the present invention, the chemical modification is a polymer.

According to an embodiment of the present invention, the polymer is selected from the group consisting of a polycationic polymer, a non-ionic water-soluble polymer, a polyether polymer and a biocompatible polymer.

According to an embodiment of the present invention, the LOX family member is selected from the group consisting of LOX, LOXL1, LOXL2, LOXL3 and LOXL4.

According to an embodiment of the present invention, the LOX family member is LOX.

According to an embodiment of the present invention, the extracellular matrix protein is encoded by a gene selected from the group consisting of ADAMTS10, ADAMTS14, ADAMTS2, ADAMTSL2, ADAMTSL4, ANGPTL4, COL17A1, COL18A1, COL1A1, COL1A2, COL3A1, COL4A1, COL4A2, COL5A1, COL6A1, COL6A3, CTGF, ECM1, EGFL7, ELN, EPHA7, FBLN5, FBN1, FGF2, FMOD, FN1, ITGA5, ITGB1, LAMB1, LAMB2, LAMC1, LTBP1, MFAP4, MFAP5, NID1, PDGFRA, SPARC, THBS1, TIMP3 and TNC.

According to an embodiment of the present invention, the extracellular matrix protein comprises a FN1 repeat domain.

According to an embodiment of the present invention, the isolated peptide comprises an amino acid sequence at least 90% identical to SEQ ID NO: 17 or SEQ ID NO: 18.

According to an embodiment of the present invention, the extracellular matrix protein is fibronectin.

According to an embodiment of the present invention, the peptide comprises at least one of the sequences selected from the group consisting of SEQ ID NO: 12 (RPKDS), SEQ ID NO: 13 (DGKTY), SEQ ID NO: 14 (WQKEY) and SEQ ID NO: 15 (ERPKDSM).

According to an embodiment of the present invention, the isolated peptide is no longer than 20 amino acids, in the absence of the modification.

According to an embodiment of the present invention, the isolated peptide is no longer than 400 amino acids.

According to an embodiment of the present invention, the disease is cancer and Duchenne muscular dystrophy (DMD).

According to an embodiment of the present invention, the disease is associated with liver fibrosis, cardiac fibrosis or lung fibrosis.

According to an embodiment of the present invention, the disease is associated with internal or external scarring.

According to an aspect of the present invention there is provided an isolated peptide comprising an amino acid sequence of a lysyl oxidase (LOX) family member target site of an extracellular matrix protein, the peptide being no longer than 50 amino acids, wherein the peptide comprises a modification which imparts the peptide with enhanced stability under physiological conditions as compared to a native form of the peptide not comprising the modification.

According to an embodiment of the present invention, the modification comprises a proteinaceous modification.

According to an aspect of the present invention there is provided a pharmaceutical composition comprising the peptide disclosed herein as the active agent and a pharmaceutically acceptable carrier.

According to an embodiment of the present invention, the composition is formulated for local delivery.

According to an embodiment of the present invention, the composition is formulated for systemic delivery.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-W. Lysyl oxidases regulate FN fibrillogenesis on soft matrices. MEF expressing FN-GFP stained for 01-integrin and LoxL3 (A). FN immunostaining, color-coded for intensity (blue—low, yellow—high), of shLOX and shControl HASMC on 1 kpa (B-C) and 40 kpa (D-E). Magnification of representative regions in (D and E). quantification of FN fibrils show significant reduction following LOX knockdown on both 1 kpa and 40 kpa (F-G, n>40). Pax-GFP on sham- and LOXL3-treated FN with or without βAPN, seeded on 1 kPa (H-M) or 0.25 kPa (P-U) gels and fixed after 1 hour (H-J, P-R) and 2 hours (K-M, S-U). Quantification of cell area show significant differences between cells seeded on treated FN regardless of rigidities (N-O, V-W, n>28). 1**p<0.0001, **p<0.001, *p<0.05, n.s.=non-significant.

FIGS. 2A-M. Lysyl oxidases stimulate adhesion initiation. Representative image of a cell on pillars and a graph of pillar displacement; the red arrowheads point to catch-and-release events. (A). Histograms of the pillar release force during catch-and-release events by cells seeded on sham or LOXL3-treated FN, demonstrating enhanced formation of 2 pN bonds upon LOXL3 treatment (B, n>243). Histogram of the maximal displacement of pillars by cells seeded on LOXL3-treated FN (C, n>243). Pax-GFP on sham- and LOXL3-treated FN with or without βAPN, seeded on glass and fixed after 15 (D-F) and 30 minutes (G-I). Quantifications of cell area show significant enhanced spreading of cells seeded on treated FN compared to controls (J-K, n>39). Histogram of focal adhesion area of cells seeded on sham- or LOXL3-treated FN demonstrating that LOXL3 treatment promotes larger adhesions (L-M). ***p<0.0001, **p<0.001, *p<0.05, n.s.=non-significant.

FIGS. 3A-R. LOX induces FN clustering. Representative dSTORM images of sham- and LOXL3-treated FN nanodomains; the curves below are the intensity values along the white lines shown in each image (A-B). dSTORM analysis of FN cluster area shows a significant increase following LOXL3 treatment (C). Representative color-coded images generated from time-lapse videos tracking fluorescence-labeled nuclei on sham- and LOXL3-treated FN (D-E). Quantifications of migration parameters show significant increases in the measured parameters of cells seeded on LOXL3-treated FN (F-H, n>426). LoxL3 and LoxL2 immunostaining of non-polar cells (I-L) and polar cells (M-P) showing that the Lox family members are localized to lamellipodial regions. 3D dSTORM image and zoom-in x-z representation of the cell edge demonstrating LoxL3 secretion (Q). Quantification of the percent of LoxL3 molecules localized below the cell leading edge (R, n=17). ***p<0.0001, **p<0.001.

FIGS. 4A-S. LOX is essential for FN fibrillogenesis. Cartoon of a FN monomer containing the oxidized lysines (marked by red asterisks) (A). Conservation of the three oxidized lysines among vertebrates (B). Cartoon representing the two FN constructs that were generated (C). Western blot against FN from lysates of cells expressing FN^{wt}, FN^{ΔRGD} and FN^{ΔOxi} (D). Phalloidin staining of FN^{WT}, FN^{ΔRGD} and FN^{ΔOxi} cells seeded on glass without external FN coating and fixed after 1 hour (E-G). Quantification of cell area shows a significant decrease in FN^{ΔOxi} cells (H, n>39). Quantification of mean fluorescence intensity of 01-integrin staining per cell shows a significant decrease in FN^{ΔOxi} cells (I, n>20). FN immunostaining, color-coded for intensity (blue—low, yellow—high), of cells expressing FN^{WT}, FN^{ΔRGD} and FN^{ΔOxi} after 24 hours of seeding on glass without external FN coating; magnification of representative regions shown below (J-L). Quantification of FN fibrils show a significant decrease in FN^{ΔOxi} cells, revealing a critical contribution of LOX family to FN fibrillogenesis (M, n>71). Representative images of cells expressing FN^{WT} and FN^{ΔOxi} after 24 hours of seeding on glass without external FN coating, stained for F-actin and FN (color-coded for intensity), showing the lack of FN fibrils even when stress fibers are assembled in FN^{ΔOxi} cells (N). Actin and FN immunostaining (color-coded for intensity) of shControl (O-P) and shLOX (Q-R) HASMC seeded on glass without external FN coating and fixed after 24 hours, demonstrating the inability of shLOX cells to form FN fibrils even when actin stress fibers are assembled. Images on the right are zoom-ins of the orange boxes in each image. Model depicting the stages (1-3) required for generating FN clusters that act as nucleators for the force-induced fibrillogenesis step (4). Created withBioRender.com. (S). ***p<0.0001, n.s.=non-significant.

FIGS. 5A-ZC. Immunofluorescence staining of cranial neural tube explants obtained from E8.5 embryos to analyze expression of LoxL3 in Tuj1-expressing neural crest cells (A). Ventral view of the maxilla of newborn wild-type (B) and LoxL3^{Δ/Δ} mouse with cleft palate (C). The palate consists of the primary palate (pp) and the secondary palate, with the hard palate (hp). LoxL3^{Δ/Δ} mouse showing complete cleft palate (arrows in C). Ventral view of the cranial base of E18.5 wild-type (D) and LoxL3^{Δ/Δ} mice (E) stained with alizarin red and alcian blue marking mineralized bone and cartilage, respectively. Maxilla (mx) is highlighted (dashed oval in E). Ap2α immunostaining of the second branchial arch artery of wild-type (F) and LoxL3^{Δ/Δ} (G) 29-30 somites mouse embryos. Quantification of the number of nuclei shows a significant reduction of Ap2α positive nuclei in LoxL3^{Δ/Δ} embryos (H, n=4 of each genotype). FN immunostaining of the border between the first and second somites of wild-type (I) and LoxL3^{Δ/Δ} (J) 26 somites embryos. Quantification shows a significant reduction in the total FN area in LoxL3^{Δ/Δ} somite border (K, n=2 of each genotype). Western blot for LOX from HASMC lysates (L). Quantification of the mean fluorescence intensity in samples immunostained for FN showing no significant difference in secreted FN levels between shContol and shLOX cells (M, n=15 images of each condition). Pax-GFP on sham- and LOXL2-treated FN with or without βAPN, seeded on 1 kPa (N-S) or 0.25 kPa (V-ZA) gels and fixed after 1 hour (N-P, V-X) and 2 hours (Q-S, Y-ZA). Quantification of cell areas show significant differences between cells seeded on treated FN regardless of rigidities (T-U, ZB-ZC, n>28). ***p<0.0001, **p<0.001, *p<0.05, n.s.=non-significant.

FIGS. 6A-V. Pax-GFP on sham- and LOXL2-treated FN, seeded on glass after 15 minutes (A-B) and 30 minutes (C-D). Quantifications of cell area show significant differences in cell area of those seeded on the treated FN (E-F, n=40). Histogram of focal adhesion area of cells seeded on sham- or LOXL2-treated FN demonstrating that LOXL2 treatment promotes larger adhesions (G-H). Paxillin immunostaining of wild-type MEFs (I-K) and human dermal fibroblasts (L-N) on sham- and LOXL3-treated FN with or without βAPN, seeded on glass and fixed after 15 minutes. Quantifications of cell area show significant differences in cell area of those seeded on the treated FN (O-P, n>38). Pax-GFP on sham- and LOXL3-treated FN followed by additional βAPN incubation on glass, fixed after 30 minutes (Q-T). Quantification of LOXL3 following treatment and washes (U, n=10). Quantification of cell area shows that no change was observed following late βAPN incubation, demonstrating that Lox activity regulates cell adhesion directly via FN rather than through integrin activation (V, n=40). ***p<0.0001, n.s.=non-significant.

FIGS. 7A-C. Sequences of the peptides identified by LC-MS/MS analysis and the lysine residues marked in red (A). FN-GFP cells seeded on FN-647 and fixed after 48 hours (B-C).

FIGS. 8A-F. Inhibitor peptides block fibronectin fibrillogenesis. Representative images of fibronectin immunostaining (color-coded for intensity) underneath human dermal fibroblast (HDF) cells exposed to the different peptides, seeded on glass without external FN coating and fixed after 1 week (A-E, scale=20 μm). A quantitative summary of the total fibronectin fibrils, demonstrating the reduction in fibrils with exposure to the different inhibitors (FN1-2 and FN1-12), as compared with the control peptides (FN1-3 and FN1-9) or the untreated sample (F).

FIGS. 9A-F. Inhibitor peptides reduce cell size. Representative images of HDF cell contour following exposure to the different peptides, seeded on glass without external FN coating and fixed after 1 week (A-E, scale=100 μm). A quantitative summary of the cell area, showing significant reduction with exposure to the different inhibitor peptides (FN1-2 and FN-1-12), as compared with the control peptides (FN1-3 and FN1-9) or the untreated sample (F).

FIGS. 10A-F. Inhibitor peptides affect cellular mechanosensation. Representative images of immunofluorescence for Yap, as a readout of mechanosensation in HDF cells following culturing with the different peptides, seeded on glass without external FN coating and fixed after 1 week (A-E, scale=100 μm). A table detailing the number of cells with nuclear or cytoplasmic Yap, as well as the percent of the nuclear protein (F).

FIGS. 11A-G. Inhibitor peptides affect cell proliferation. Representative images of lung fibroblasts following 72 hours in culture without peptide addition (A), control peptide (FN1-9; B) or inhibitor peptides (FN1-2, FN1-12 or both of them; C-E, respectively). Quantification of culture confluency following 72 hours demonstrates the inhibitor peptides (FN1-2 and FN1-12) or both (mix) but not with the control peptide (FN1-9) inhibit cell proliferation (F). Note the lack of effect in the first day of culturing (G).

FIG. 12. Inhibitor peptides undergo oxidation by Lysyl oxidases. Oxidation assay demonstrates the inhibitor peptides (FN1-2 and FN1-12) but not the control peptides (FN1-3 and FN1-9) undergo oxidation by LOXL3. This oxidation is inhibited following the addition of βAPN (beta aminopropionitrile, LOX family competitive inhibitor).

FIGS. 13A-E. Inhibitor Fc-fused peptides affect fibronectin fibrillogenesis. Representative images of fibronectin immunostaining (color-coded for intensity) underneath human dermal fibroblasts (HDF) seeded on glass without external FN coating and fixed after 1 week; the cells were either left without Fc-fused peptide addition (A), with Fc-fused inhibitor peptide FN1-2 (B), or with Fc-fused inhibitor peptide FN1-2 mutated at the oxidation site 116 (lys-to-arg) (C). Boxplot representing the ratio between the total area of fibronectin fibrils longer than 5 μm and the total measured area in each image. This boxplot shows the significant reduction in the percentage of long fibrils when cells are exposed to the Fc-fused inhibitor peptides (D). Boxplots of the mean area of fibrils in each condition, demonstrating the significant decrease in fibrils length following exposure to the Fc-fused inhibitor peptides (E).

FIGS. 14A-C. Inhibitor Fc-fused peptides affect cell proliferation. Representative images of normal human lung fibroblasts (NHLF) following 48 hours in culture without Fc-fused peptide addition (A) or with Fc-fused inhibitor peptide FN1-2 (B). Quantification of culture confluency over time demonstrates the inhibitor Fc-fused peptide FN1-2 inhibits cell proliferation (C).

### DESCRIPTION OF SPECIFIC EMBODIMENTS OF THE INVENTION

The present invention, in some embodiments thereof, relates to peptides for the treatment of diseases associated with fibrosis.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Fibrosis is the abnormal accumulation of fibrous extracellular matrix which comes at the expense of cells within the affected tissue, thereby leading to gradual deterioration and malfunction of the tissue. An essential early step in the fibrotic process is fibrillogenesis of the extracellular matrix protein, fibronectin. The present inventors have now found that fibronectin fibrillogenesis depends on its oxidation at specific lysine residues by members of the lysyl oxidase (LOX) enzyme family.

Accordingly, the present inventors propose a novel strategy to inhibit fibrosis by using short peptides from fibronectin (or other Lox substrates) that will serve as baits for lysyl oxidases, thereby inhibiting oxidation of full-length fibronectin proteins and blocking the fibrotic reaction.

Thus, according to a first aspect of the present invention, there is provided an isolated peptide comprising an amino acid sequence of a member of a lysyl oxidase (LOX) family target site of an extracellular matrix protein, the peptide being no longer than 50 amino acids, for use in treating a disease associated with fibrosis in a subject in need thereof.

Lysyl oxidase family members (LOX and LOXL1 [lysyl oxidase-like 1], LOXL2 [lysyl oxidase-like 2], LOXL3 [lysyl oxidase-like 3], and LOXL4 [lysyl oxidase like 4]) are extracellular copper-dependent enzymes that play a key role in ECM cross-linking, but have also other intracellular functions relevant to fibrosis and carcinogenesis.

As used herein “Lysyl Oxidase” or “LOX” refers to the protein product of the LOX gene. In humans, the LOX gene is located on chromosome 5q23.3-31.2. The primary sequence of the LOX protein is highly conserved in mammals. Human LOX is synthesized as a pre-proprotein (pre-pro-LOX) of 417 amino acids (UniProtKB-P28300, which undergoes a number of post-translational modifications within the endoplasmic reticulum (ER) and post-ER e.g., glycosylation as described below. After cleavage of the 21 amino acid signal sequence, the N-terminal propeptide, comprising 147 amino acid residues, is N-glycosylated and the C-terminal sequence containing the 249 amino acid residue mature protein which is also referred to herein as the part which comprises the LOX catalytic activity, is distinctively folded to acquire at least three disulfide bonds. Copper is a cofactor of the functional catalyst, incorporated into the nascent enzyme within the ER. The enzyme also contains a peptidyl organic cofactor, lysyltyrosine quinone (LTQ) generated by an intramolecular cross-link between lysine 320 and the copper-dependent oxidation product of tyrosine 355.

As used herein “fibrosis” refers to the accumulation of extracellular matrix constituents that occurs following trauma, inflammation, tissue repair, immunological reactions, cellular hyperplasia, and neoplasia. Examples of tissue fibrosis include, but are not limited to, pulmonary fibrosis, renal fibrosis, cardiac fibrosis, cirrhosis and fibrosis of the liver, skin scars and keloids, adhesions, fibromatosis, atherosclerosis, and amyloidosis.

In some embodiments, the fibrotic condition is primary fibrosis. In some embodiments, the fibrotic condition is idiopathic. In some embodiments, the fibrotic condition is associated with (e.g., is secondary to) a disease (e.g., an infectious disease, an inflammatory disease, an autoimmune disease, a malignant or cancerous disease, and/or a connective disease); a toxin; an insult (e.g., an environmental hazard (e.g., asbestos, coal dust, polycyclic aromatic hydrocarbons), cigarette smoking, a wound); a medical treatment (e.g., surgical incision, chemotherapy or radiation), or a combination thereof.

In some embodiments, the fibrotic condition is a fibrotic condition of the lung, a fibrotic condition of the liver, a fibrotic condition of the heart or vasculature, a fibrotic condition of the kidney, a fibrotic condition of the skin, a fibrotic condition of the gastrointestinal tract, a fibrotic condition of the bone marrow or a hematopoietic tissue, a fibrotic condition of the nervous system, or a combination thereof. In some embodiments, the fibrotic condition affects a tissue chosen from one or more of muscle, tendon, cartilage, skin (e.g., skin epidermis or endodermis), cardiac tissue, vascular tissue (e.g., artery, vein), pancreatic tissue, lung tissue, liver tissue, kidney tissue, uterine tissue, ovarian tissue, neural tissue, testicular tissue, peritoneal tissue, colon, small intestine, biliary tract, gut, bone marrow, or hematopoietic tissue.

In some embodiments, the fibrotic condition is a fibrotic condition of the lung. In some embodiments, the fibrotic condition of the lung is chosen from one or more of: pulmonary fibrosis, idiopathic pulmonary fibrosis (IPF), usual interstitial pneumonitis (UIP), interstitial lung disease, cryptogenic fibrosing alveolitis (CFA), bronchiolitis obliterans, or bronchiectasis. In some embodiments, the fibrosis of the lung is secondary to a disease, a toxin, an insult, a medical treatment, or a combination thereof. In some embodiments, fibrosis of the lung is associated with one or more of: a disease process such as asbestosis and silicosis; an occupational hazard; an environmental pollutant; cigarette smoking; an autoimmune connective tissue disorders (e.g., rheumatoid arthritis, scleroderma and systemic lupus erythematosus (SLE)); a connective tissue disorder such as sarcoidosis; an infectious disease, e.g., infection, particularly chronic infection; a medical treatment, including but not limited to, radiation therapy, and drug therapy, e.g., chemotherapy (e.g., treatment with as bleomycin, methotrexate, amiodarone, busulfan, and/or nitrofurantoin). In some embodiments, the fibrotic condition of the lung treated with the methods of the invention is associated with (e.g., secondary to) a cancer treatment, e.g., treatment of a cancer (e.g. squamous cell carcinoma, testicular cancer, Hodgkin's disease with bleomycin). In some embodiments, the fibrotic condition is a fibrotic condition of the liver. In certain embodiments, the fibrotic condition of the liver is chosen from one or more of: fatty liver disease, steatosis (e.g., nonalcoholic steatohepatitis (NASH), cholestatic liver disease (e.g., primary biliary cirrhosis (PBC), cirrhosis, alcohol-induced liver fibrosis, biliary duct injury, biliary fibrosis, cholestasis or cholangiopathies. In some embodiments, hepatic or liver fibrosis includes, but is not limited to, hepatic fibrosis associated with alcoholism, viral infection, e.g., hepatitis (e.g., hepatitis C, B or D), autoimmune hepatitis, non-alcoholic fatty liver disease (NAFLD), progressive massive fibrosis, exposure to toxins or irritants (e.g., alcohol, pharmaceutical drugs and environmental toxins).

In some embodiments, the fibrotic condition is a fibrotic condition of the heart. In certain embodiments, the fibrotic condition of the heart is myocardial fibrosis (e.g., myocardial fibrosis associated with radiation myocarditis, a surgical procedure complication (e.g., myocardial post-operative fibrosis), infectious diseases (e.g., Chagas disease, bacterial, trichinosis or fungal myocarditis)); granulomatous, metabolic storage disorders (e.g., cardiomyopathy, hemochromatosis); developmental disorders (e.g., endocardial fibroelastosis); arteriosclerotic, or exposure to toxins or irritants (e.g., drug induced cardiomyopathy, drug induced cardiotoxicity, alcoholic cardiomyopathy, cobalt poisoning or exposure). In some embodiments, the myocardial fibrosis is associated with an inflammatory disorder of cardiac tissue (e.g., myocardial sarcoidosis).

In some embodiments, the fibrotic condition is a fibrotic condition of the kidney. In some embodiments, the fibrotic condition of the kidney is chosen from one or more of: renal fibrosis (e.g., chronic kidney fibrosis), nephropathies associated with injury/fibrosis (e.g., chronic nephropathies associated with diabetes (e.g., diabetic nephropathy)), lupus, scleroderma of the kidney, glomerular nephritis, focal segmental glomerular sclerosis, IgA nephropathyrenal fibrosis associated with human chronic kidney disease (CKD), chronic progressive nephropathy (CPN), tubulointerstitial fibrosis, ureteral obstruction, chronic uremia, chronic interstitial nephritis, radiation nephropathy, glomerulosclerosis, progressive glomerulonephrosis (PGN), endothelial/thrombotic microangiopathy injury, HIV-associated nephropathy, or fibrosis associated with exposure to a toxin, an irritant, or a chemotherapeutic agent.

In some embodiments, the fibrotic condition is a fibrotic condition of the skin. In some embodiments, the fibrotic condition of the skin is chosen from one or more of: skin fibrosis, scleroderma, nephrogenic systemic fibrosis (e.g., resulting after exposure to gadolinium which is frequently used as a contrast substance for MRIs in patients with severe kidney failure), scarring and keloid.

In some embodiments, the fibrotic condition is a fibrotic condition of the gastrointestinal tract. In some embodiments, the fibrotic condition is chosen from one or more of fibrosis associated with scleroderma; radiation induced gut fibrosis; fibrosis associated with a foregut inflammatory disorder such as Barrett's esophagus and chronic gastritis, and/or fibrosis associated with a hindgut inflammatory disorder, such as inflammatory bowel disease (IBD), ulcerative colitis and Crohn's disease.

In some embodiments, the fibrotic condition is adhesions. In some embodiments, the adhesions are chosen from one or more of: abdominal adhesions, peritoneal adhesions, pelvic adhesions, pericardial adhesions, peridural adhesions, peritendinous or adhesive capsulitis.

In some embodiments, the fibrotic condition is a fibrotic condition of the eye. In some embodiments, the fibrotic condition of the eye involves diseases of the anterior segment of the eye such as glaucoma and corneal opacification; in some embodiments, the fibrotic condition of the eye involves disease of the posterior segment of the eye such as age-related macular degeneration, diabetic retinopathy, retinopathy of prematurity and neovascular glaucoma; in some embodiments, the fibrotic condition of the eye results from fibrosis following ocular surgery.

In some embodiments, the fibrotic condition is a fibrotic condition of the bone marrow or a hematopoietic tissue. In some embodiments, the fibrotic condition of the bone marrow is an intrinsic feature of a chronic myeloproliferative neoplasm of the bone marrow, such as primary myelofibrosis (also referred to herein as angiogenic myeloid metaplasia or chronic idiopathic myelofibrosis). In some embodiments, the bone marrow fibrosis is associated with (e.g., is secondary to) a malignant condition or a condition caused by a clonal proliferative disease. In some embodiments, the bone marrow fibrosis is associated with a hematologic disorder (e.g., a hematologic disorder chosen from one or more of polycythemia vera, essential thrombocythemia, myelodysplasia, hairy cell leukemia, lymphoma (e.g., Hodgkin or non-Hodgkin lymphoma), multiple myeloma or chronic myelogeneous leukemia (CIVIL)). In some embodiments, the bone marrow fibrosis is associated with (e.g., secondary to) a non-hematologic disorder (e.g., a non-hematologic disorder chosen from solid tumor metastasis to bone marrow, an autoimmune disorder (e.g., systemic lupus erythematosus, scleroderma, mixed connective tissue disorder, or polymyositis), an infection (e.g., tuberculosis), or secondary hyperparathyroidism associated with vitamin D deficiency.

According to a specific embodiment, the fibrotic condition is of the muscle.

As used herein “treating” refers to abrogating, substantially inhibiting, slowing or reversing the progression of a condition, substantially ameliorating clinical or aesthetical symptoms of a condition or substantially preventing the appearance of clinical or aesthetical symptoms of a condition.

As used herein “subject” refers to a subject diagnosed with or at risk of fibrosis.

The peptide agents disclosed herein do not naturally occur in nature, either because they are isolated from a natural environment thereof e.g., the human or animal body, or because they are mutated with respect to the wild-type form or because they modified e.g., attached to a heterologous moiety e.g., protein or chemical.

The peptide agents may be derived from (e.g. may be comprised in, or fragments of) extracellular matrix proteins that are known to comprise target sites for members of the LOX family (LOX, LOX1, LOX2, LOX3 or LOX4). In one embodiment, the target site is a binding site. In another embodiment, the target site is an oxidation site.

Examples of extracellular matrix (ECM) proteins include ADAMTS10, ADAMTS14, ADAMTS2, ADAMTSL2, ADAMTSL4, ANGPTL4, COL17A1, COL18A1, COL1A1, COL1A2, COL3A1, COL4A1, COL4A2, COL5A1, COL6A1, COL6A3, CTGF, ECM1, EGFL7, ELN, EPHA7, FBLN5, FBN1, FGF2, FMOD, FN1, ITGA5, ITGB1, LAMB1, LAMB2, LAMC1, LTBP1, MFAP4, MFAP5, NID1, PDGFRA, SPARC, THBS1, TIMP3 and TNC.

According to a particular embodiment, the target site is comprised in an FN1 domain of an ECM protein.

The term “FN1 domain” refers to an amino acid sequence of 30-50 amino acids (e.g. 40), primarily found in fibronectin, as well as in other proteins. Its structure contains 2 anti-parallel beta sheets, the first a double stranded one linked by a disulfide bond to a triple stranded sheet. The second disulfide bond links the C-terminal of each sheet.

Amino acid sequences of examples of FN1 domains are provided in SEQ ID NOs: 32-46.

Examples of proteins having an FN1 domain include F12; FN1; HGFAC and PLAT.

According to a particular embodiment, the peptide comprises a sequence of a LOX target site of fibronectin (FN1).

The amino acid sequence of fibronectin is set forth in SEQ ID NO: 16.

The amino acid sequence of the peptide agents may be identical to a fragment of the ECM protein that comprises the target sites or may be at least 90%, 91%, 92%, 93%, 94%, 95%, 96%, 97%, 98%, 99% or 100% identical to the fragment of the ECM protein (e.g. fibronectin) that comprises the target site.

The amino acids of the peptides of some embodiments of the present invention may be substituted either conservatively or non-conservatively. Preferably, the substitution does not take place in the LOX binding site of the extracellular matrix protein (e.g. fibronectin). Even more specifically, the substitution is preferably not on the lysine residue of the LOX binding site.

Thus, the sequence of the peptide may be at least 90%, 91%, 92%, 93%, 94%, 95%, 96%, 97%, 98%, 99% or 100% identical to SEQ ID NO: 17 or 18.

In another embodiment, the sequence of the peptide may be at least 90%, 91%, 92%, 93%, 94%, 95%, 96%, 97%, 98%, 99% or 100% identical to SEQ ID NO: 19 or 20.

The term “conservative substitution” as used herein, refers to the replacement of an amino acid present in the native sequence in the polypeptide with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

As naturally occurring amino acids are typically grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(˜CH2)5COOH]—CO— for aspartic acid. Those non-conservative substitutions which fall under the scope of the present invention are those which still constitute a peptide which prevent oxidation of full-length fibronectin proteins.

In one embodiment, the peptide agents comprise at least one of the following sequences: SEQ ID NO: 12 (RPKDS), SEQ ID NO: 13 (DGKTY), SEQ ID NO: 14 (WQKEY) or SEQ ID NO: 15 (ERPKDSM).

The sequences derived from the ECM proteins may be less than 50 amino acids in length, less than 40 amino acids in length, less than 30 amino acids in length, less than 20 amino acids in length or even less than 10, 9, 8, 7 or 6 amino acids in length.

The term “peptide” as used herein refers to a polymer of natural or synthetic amino acids (preferably no longer than 100 or 50 amino acids in length).

The peptide may include modifications such as, but are not limited to N terminus modification, C terminus modification, polypeptide bond modification, including, but not limited to, CH2—NH, CH2—S, CH2—S═O, O═C—NH, CH2—O, CH2—CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided herein under.

Polypeptide bonds (—CO—NH—) within the polypeptide may be substituted, for example, by N-methylated bonds (—N(CH3)—CO—), ester bonds (—C(R)H—C—O—O—C(R)—N—), ketomethylen bonds (—CO—CH2—), α-aza bonds (—NH—N(R)—CO—), wherein R is any alkyl, e.g., methyl, carba bonds (—CH2—NH—), hydroxyethylene bonds (—CH(OH)—CH2—), thioamide bonds (—CS—NH—), olefinic double bonds (—CH═CH—), retro amide bonds (—NH—CO—), polypeptide derivatives (—N(R)—CH2—CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc.).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers).

Tables A and B below list naturally occurring amino acids (Table A) and non-conventional or modified amino acids (Table B) which can be used with the present invention.

TABLE A

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as | Xaa | X |
| above | | |

TABLE B

| Non-conventional | | Non-conventional | |
|---|---|---|---|
| amino acid | Code | amino acid | Code |
| α-aminobutyric acid | Abu | L-N-methylalanine | Nmala |
| α-amino-α- | Mgabu | L-N-methylarginine | Nmarg |
| methylbutyrate | | | |
| aminocyclopropane- | Cpro | L-N-methylasparagine | Nmasn |
| Carboxylate | | L-N-methylaspartic | Nmasp |
| | | acid | |
| aminoisobutyric acid | Aib | L-N-methylcysteine | Nmcys |
| aminonorbornyl- | Norb | L-N-methylglutamine | Nmgin |
| Carboxylate | | L-N-methylglutamic | Nmglu |
| | | acid | |
| Cyclohexylalanine | Chexa | L-N-methylhistidine | Nmhis |
| Cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N- | Nmphe |
| | | methylphenylalanine | |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t- | Nmtbug |
| | | butylglycine | |
| D-threonine | Dthr | L-norleucine | Nle |
| D-tryptophan | Dtrp | L-norvaline | Nva |
| D-tyrosine | Dtyr | α-methyl- | Maib |
| | | aminoisobutyrate | |
| D-valine | Dval | α-methyl-γ- | Mgabu |
| | | aminobutyrate | |
| D-α-methylalanine | Dmala | α | Mchexa |
| | | ethylcyclohexylalanine | |
| D-α-methylarginine | Dmarg | α- | Mcpen |
| | | methylcyclopentylalanine | |
| D-α-methylasparagine | Dmasn | α-methyl-α- | Manap |
| | | napthylalanine | |
| D-α-methylaspartate | Dmasp | α-methylpenicillamine | Mpen |
| D-α-methylcysteine | Dmcys | N-(4- | Nglu |
| | | aminobutyl)glycine | |
| D-α-methylglutamine | Dmgln | N-(2- | Naeg |
| | | aminoethyl)glycine | |
| D-α-methylhistidine | Dmhis | N-(3- | Norn |
| | | aminopropyl)glycine | |
| D-α-methylisoleucine | Dmile | N-amino-α- | Nmaabu |
| | | methylbutyrate | |
| D-α-methylleucine | Dmleu | α-napthylalanine | Anap |
| D-α-methyllysine | Dmlys | N-benzylglycine | Nphe |
| D-α-methylmethionine | Dmmet | N-(2- | Ngln |
| | | carbamylethyl)glycine | |
| D-α-methylornithine | Dmorn | N- | Nasn |
| | | (carbamylmethyl)glycine | |
| D-α- | Dmphe | N-(2- | Nglu |
| methylphenylalanine | | carboxyethyl)glycine | |
| D-α-methylproline | Dmpro | N- | Nasp |
| | | (carboxymethyl)glycine | |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2- | Nbhm |
| | | diphenylethyl)glycine | |
| D-α-methylcysteine | Dnmcys | N-(3,3- | Nbhe |
| | | diphenylpropyl)glycine | |
| D-N-methylleucine | Dnmleu | N-(3-indolylyethyl) | Nhtrp |
| | | glycine | |
| D-N-methyllysine | Dnmlys | N-methyl-γ- | Nmgabu |
| | | aminobutyrate | |
| N- | Nmchexa | D-N-methylmethionine | Dnmmet |
| methylcyclohexylalanine | | | |
| D-N-methylornithine | Dnmorn | N- | Nmcpen |
| | | methylcyclopentylalanine | |
| N-methylglycine | Nala | D-N- | Dnmphe |
| | | methylphenylalanine | |
| N- | Nmaib | D-N-methylproline | Dnmpro |
| methylaminoisobutyrate | | | |
| N-(1- | Nile | D-N-methylserine | Dnmser |
| methylpropyl)glycine | | | |
| N-(2- | Nile | D-N-methylserine | Dnmser |
| methylpropyl)glycine | | | |
| N-(2- | Nleu | D-N-methylthreonine | Dnmthr |
| methylpropyl)glycine | | | |
| D-N-methyltryptophan | Dnmtrp | N-(1- | Nva |
| | | methylethyl)glycine | |
| D-N-methyltyrosine | Dnmtyr | N-methyla- | Nmanap |
| | | napthylalanine | |
| D-N-methylvaline | Dnmval | N-methylpenicillamine | Nmpen |
| γ-aminobutyric acid | Gabu | N-(p- | Nhtyr |
| | | hydroxyphenyl)glycine | |
| L-t-butylglycine | Tbug | N-(thiomethyl)glycine | Ncys |
| L-ethylglycine | Etg | Penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t- | Mtbug |
| | | butylglycine | |
| L-α-methylcysteine | Mcys | L-methylethylglycine | Metg |
| L-α thylglutamine | Mgln | L-α-methylglutamate | Mglu |
| L-α-methylhistidine | Mhis | L-α-methylhomo | Mhphe |
| | | phenylalanine | |
| L-α-methylisoleucine | Mile | N-(2- | Nmet |
| | | methylthioethyl)glycine | |
| D-N-methylglutamine | Dnmgln | N-(3- | Narg |
| | | guanidinopropyl)glycine | |
| D-N-methylglutamate | Dnmglu | N-(1- | Nthr |
| | | hydroxyethyl)glycine | |
| D-N-methylhistidine | Dnmhis | N- | Nser |
| | | (hydroxyethyl)glycine | |
| D-N-methylisoleucine | Dnmile | N- | Nhis |
| | | (imidazolylethyl)glycine | |
| D-N-methylleucine | Dnmleu | N-(3- | Nhtrp |
| | | indolylyethyl)glycine | |
| D-N-methyllysine | Dnmlys | N-methyl-γ- | Nmgabu |
| | | aminobutyrate | |
| N- | Nmchexa | D-N-methylmethionine | Dnmmet |
| methylcyclohexylalanine | | | |
| D-N-methylornithine | Dnmorn | N- | Nmcpen |
| | | methylcyclopentylalanine | |
| N-methylglycine | Nala | D-N- | Dnmphe |
| | | methylphenylalanine | |
| N- | Nmaib | D-N-methylproline | Dnmpro |
| methylaminoisobutyrate | | | |
| N-(1- | Nile | D-N-methylserine | Dnmser |
| methylpropyl)glycine | | | |
| N-(2- | Nleu | D-N-methylthreonine | Dnmthr |
| methylpropyl)glycine | | | |
| D-N-methyltryptophan | Dnmtrp | N-(1- | Nval |
| | | methylethyl)glycine | |
| D-N-methyltyrosine | Dnmtyr | N-methyla- | Nmanap |
| | | napthylalanine | |
| D-N-methylvaline | Dnmval | N-methylpenicillamine | Nmpen |
| γ-aminobutyric acid | Gabu | N-(p- | Nhtyr |
| | | hydroxyphenyl)glycine | |
| L-t-butylglycine | Tbug | N-(thiomethyl)glycine | Ncys |
| L-ethylglycine | Etg | Penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t- | Mtbug |
| | | butylglycine | |
| L-α-methylcysteine | Mcys | L-methylethylglycine | Metg |
| L-α-methylglutamine | Mgln | L-α-methylglutamate | Mglu |
| L-α ethylhistidine | Mhis | L-α- | Mhphe |
| | | methylhomophenylalanine | |
| L-α thylisoleucine | Mile | N-(2- | Nmet |
| | | methylthioethyl)glycine | |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α | Mphe | L-α-methylproline | Mpro |
| methylphenylalanine | | | |
| L-α-methylserine | Mser | L-α-methylthreonine | Mthr |
| L-α ethylvaline | Mtrp | L-α-methyltyrosine | Mtyr |
| L-α-methylleucine | Mval | L-N- | Nmhphe |
| | Nnbhm | methylhomophenylalanine | |
| N-(N-(2,2- | | N-(N-(3,3- | |
| diphenylethyl) | | diphenylpropyl) | |
| carbamylmethyl-glycine | Nnbhm | carbamylmethyl(1) | Nnbhe |
| | | glycine | |
| 1-carboxy-1-(2,2- | Nmbc | | |
| diphenyl | | | |
| ethylamino) | | | |
| cyclopropane | | | |

The proteinaceous modification can be attached to the polypeptide by ways of chemical attachment (fusion polypeptide such as by the use of linkers and/or active groups) or by recombinant DNA technology, whereby the synthetic polypeptide is a chimeric polypeptide.

According to another embodiment, the peptide comprises a protecting moiety, a solubility enhancing moiety and/or a stabilizing moiety.

For example, the peptide may comprise a modification which imparts the peptide with enhanced stability under physiological conditions as compared to a native form of the peptide not comprising the modification.

Methods of determining stability are well known in the art.

As used herein “stability” refers to at least thermal stability. The method is based on measuring ultra-high-resolution protein stability using intrinsic tryptophan or tyrosine fluorescence.

As used herein “enhanced” or “increased” refers to an increase by at least 10%, 20%, 30%, 50%, 60%, 70%, 80%, 90% or more, say 100%, with respect to that of the native peptide.

The term “protecting moiety” refers to any moiety (e.g. chemical moiety) capable of protecting the polypeptide from adverse effects such as proteolysis, degradation or clearance, or alleviating such adverse effects.

The term “stabilizing moiety” refers to any moiety (e.g. chemical moiety) that inhibits or prevents a polypeptide from degradation.

The addition of a protecting moiety or a stabilizing moiety to the polypeptide typically results in masking the charge of the polypeptide terminus, and/or altering chemical features thereof, such as, hydrophobicity, hydrophilicity, reactivity, solubility and the like. Examples of suitable protecting moieties can be found, for example, in Green et al., “Protective Groups in Organic Chemistry”, (Wiley, 2.sup.nd ed. 1991) and Harrison et al., “Compendium of Synthetic Organic Methods”, Vols. 1-8 (John Wiley and Sons, 1971-1996).

The protecting moiety (or group) or stabilizing moiety (or group) may be added to the N-(amine) terminus and/or the C-(carboxyl) terminus of the polypeptide.

Representative examples of N-terminus protecting/stabilizing moieties include, but are not limited to, formyl, acetyl (also denoted herein as “Ac”), trifluoroacetyl, benzyl, benzyloxycarbonyl (also denoted herein as “CBZ”), tert-butoxycarbonyl (also denoted herein as “BOC”), trimethylsilyl (also denoted “TMS”), 2-trimethylsilyl-ethanesulfonyl (also denoted “SES”), trityl and substituted trityl groups, allyloxycarbonyl, 9-fluorenylmethyloxycarbonyl (also denoted herein as “FMOC”), nitro-veratryloxycarbonyl (also denoted herein as “NVOC”), t-amyloxycarbonyl, adamantyl-oxycarbonyl, and p-methoxybenzyloxycarbonyl, 2-chlorobenzyloxycarbonyl and the like, nitro, tosyl (CH3C6H4SO2—), adamantyloxycarbonyl, 2,2,5,7,8-pentamethylchroman-6-sulfonyl, 2,3,6-trimethyl-4-methoxyphenylsulfonyl, t-butyl benzyl (also denoted herein as “BZL”) or substituted BZL, such as, p-methoxybenzyl, p-nitrobenzyl, p-chlorobenzyl, o-chlorobenzyl, 2,6-dichlorobenzyl, t-butyl, cyclohexyl, cyclopentyl, benzyloxymethyl (also denoted herein as “BOM”), tetrahydropyranyl, chlorobenzyl, 4-bromobenzyl, and 2,6-dichlorobenzyl.

According to one embodiment of the invention, the protecting/stabilizing moiety is an amine protecting moiety.

According to a specific embodiment, the protecting/stabilizing moiety is a terminal cysteine residue.

Representative examples of C-terminus protecting/stabilizing moieties are typically moieties that lead to acylation of the carboxy group at the C-terminus and include, but are not limited to, benzyl and trityl ethers as well as alkyl ethers, tetrahydropyranyl ethers, trialkylsilyl ethers, allyl ethers, monomethoxytrityl and dimethoxytrityl. Alternatively the —COOH group of the C-terminus may be modified to an amide group.

Other modifications of polypeptides include replacement of the amine and/or carboxyl with a different moiety, such as hydroxyl, thiol, halide, alkyl, aryl, alkoxy, aryloxy and the like.

According to a specific embodiment, the protecting/stabilizing moiety is an amide.

According to a specific embodiment, the protecting/stabilizing moiety is a terminal cysteine residue.

According to one embodiment, the protecting/stabilizing moiety comprises at least one, two, three or more cysteine residues at the N- or C-termini of the polypeptide.

Also included in the scope of the present invention are “chemical derivative” of a polypeptide or analog. Such chemical derivates contain additional chemical moieties not normally a part of the polypeptide. Covalent modifications of the polypeptide are included within the scope of this invention. Such modifications may be introduced into the molecule by reacting targeted amino acid residues of the polypeptide with an organic derivatizing agent that is capable of reacting with selected side chains or terminal residues. Many such chemical derivatives and methods for making them are well known in the art, some are discussed hereinbelow.

Also included in the scope of the invention are salts of the peptides and analogs of the invention. As used herein, the term “salts” refers to both salts of carboxyl groups and to acid addition salts of amino groups of the polypeptide molecule. Salts of a carboxyl group may be formed by means known in the art and include inorganic salts, for example, sodium, calcium, ammonium, ferric or zinc salts, and the like, and salts with organic bases such as those formed for example, with amines, such as triethanolamine, arginine, or lysine, piperidine, procaine, and the like. Acid addition salts include, for example, salts with mineral acids such as, for example, hydrochloric acid or sulfuric acid, and salts with organic acids, such as, for example, acetic acid or oxalic acid. Such chemical derivatives and salts are preferably used to modify the pharmaceutical properties of the polypeptide insofar as stability, solubility, etc., are concerned.

According to one embodiment of the invention, the peptide (capable of inhibiting oxidation of full-length fibronectin proteins and/or blocking the fibrotic reaction) is attached to a heterologous moiety.

As used herein the phrase “heterologous moiety” refers to an amino acid sequence which does not endogenously form a part of the isolated polypeptide's amino acid sequence. Preferably, the heterologous moiety does not affect the biological activity of the isolated polypeptide (e.g. inhibiting oxidation of full-length fibronectin proteins and blocking the fibrotic reaction).

The heterologous moiety may thus serve to ensure stability of the isolated peptide of the present invention without compromising its activity. For example, the heterologous polypeptide may increase the half-life of the isolated peptide or molecule in the serum.

The heterologous moiety of the present invention may be capable of inducing an antibody dependent cellular-mediated cytotoxicity (ADCC) response.

According to one embodiment, the heterologous moiety does not induce an immune response. Thus, for instance, in the case of Ig, it may contain human sequences that do not produce an immune response in a subject administered therewith.

According to one embodiment, the heterologous moiety is for increasing avidity of the peptide.

According to one embodiment, the heterologous moiety is for multimerization of the isolated peptide (e.g. at least for dimerization of the isolated peptides.

According to one embodiment, the heterologous moiety is a proteinaceous moiety. For this embodiment, the total length of the peptide (together with the heterologous proteinaceous moiety) may be less than 500 amino acids in length, less than 400 amino acids in length, less than 300 amino acids in length, less than 200 amino acids in length, less than 100 amino acids in length, less than 50 amino acids in length or even less than 40, 30 or 20 amino acids in length (wherein the length of the peptide derived from the ECM protein and having the biological activity is typically less than 50 amino acids in length).

Examples of heterologous amino acid sequences that may be used in accordance with the teachings of the present invention include, but are not limited to, immunoglobulin, galactosidase, glucuronidase, glutathione-S-transferase (GST), carboxy terminal polypeptide (CTP) from chorionic gonadotrophin (CGb) and chloramphenicol acetyltransferase (CAT) [see for example U.S. Publication No. 20030171551].

According to a specific embodiment, the heterologous amino acid sequence is an immunoglobulin sequence.

For example, the peptide may be fused to the Fc domain of a human IgG (as referred to herein in one embodiment Fc-peptide). In particular aspects, the immunoglobulin fusion includes the hinge, CH2 and CH3, or the hinge, CH1, CH2 and CH3 regions of an IgG1 molecule. According to a specific embodiment, the Fc is as set forth in SEQ ID NO: 27. For the production of immunoglobulin fusions see also U.S. Pat. No. 5,428,130. The Fc moiety can be derived from mouse IgG1 or human IgG2_{M}4. Human IgG2_{M}4 (See U.S. Published Application No. 20070148167 and U.S. Published Application No. 20060228349) is an antibody from IgG2 with mutations with which the antibody maintains normal pharmacokinetic profile but does not possess any known effector function.

It will be appreciated that the stability enhancing moiety may be attached directly to the peptide or via a linker (e.g. 1, 2, 3, 4, or more amino acids).

Exemplary peptides attached to Fc moieties contemplated by the present invention are provided in SEQ ID NOs: 28 and 29.

Fusion proteins further include the peptide fused to human serum albumin, transferrin, or an antibody.

In further still aspects, the peptide is conjugated to a carrier protein such as human serum albumin, transferrin, or an antibody molecule.

Generally the heterologous amino acid sequence is localized at the amino- or carboxyl-terminus (N-ter or C-ter, respectively) of the isolated polypeptide of the present invention. The heterologous amino acid sequence may be attached to the isolated polypeptide amino acid sequence by any of polypeptide or non-polypeptide bond. Attachment of the isolated polypeptide amino acid sequence to the heterologous amino acid sequence may be effected by direct covalent bonding (polypeptide bond or a substituted polypeptide bond) or indirect binding such as by the use of a linker having functional groups. Functional groups include, without limitation, a free carboxylic acid (C(═O)OH), a free amino group (NH₂), an ester group (C(═O)OR, where R is alkyl, cycloalkyl or aryl), an acyl halide group (C(═O)A, where A is fluoride, chloride, bromide or iodide), a halide (fluoride, chloride, bromide or iodide), a hydroxyl group (OH), a thiol group (SH), a nitrile group (C-N), a free C-carbamic group (NR″—C(═O)—OR′, where each of R′ and R″ is independently hydrogen, alkyl, cycloalkyl or aryl).

In yet another embodiment, the peptides comprise moieties which improve solubility. For example, a series of lysine residues. Thus, for example, the following peptides with solubility moieties are contemplated—SEQ ID NOs: 25 and 26.

The peptides of the present invention can be generated using recombinant techniques such as described by Bitter et al. (1987) Methods in Enzymol. 153:516-544; Studier et al. (1990) Methods in Enzymol. 185:60-89; Brisson et al. (1984) Nature 310:511-514; Takamatsu et al. (1987) EMBO J. 6:307-311; Coruzzi et al. (1984) EMBO J. 3:1671-1680; Brogli et al. (1984) Science 224:838-843; Gurley et al. (1986) Mol. Cell. Biol. 6:559-565 and Weissbach & Weissbach, 1988, Methods for Plant Molecular Biology, Academic Press, NY, Section VIII, pp 421-463.

The heterologous moiety may also be chemically linked to the isolated peptide following the independent generation of each. Thus, the two polypeptides may be covalently or non-covalently linked using any linking or binding method and/or any suitable chemical linker known in the art. Such linkage can be direct or indirect, as by means of a polypeptide bond or via covalent bonding to an intervening linker element, such as a linker polypeptide or other chemical moiety, such as an organic polymer. Such chimeric peptides may be linked via bonding at the carboxy (C) or amino (N) termini of the peptides, or via bonding to internal chemical groups such as straight, branched, or cyclic side chains, internal carbon or nitrogen atoms, and the like. The exact type and chemical nature of such cross-linkers and cross linking methods is preferably adapted to the type and nature of the peptides used.

Thus, the peptide of this aspect of the present invention may comprise a heterologous moiety, as described above. Additionally or alternatively, the peptide amino acid sequence of the present invention may be attached to a non-proteinaceous moiety.

The phrase “non-proteinaceous moiety” as used herein refers to a molecule, not including polypeptide bonded amino acids, that is attached to the above-described isolated polypeptide's amino acid sequence.

According to one embodiment, the non-proteinaceous moiety is non-toxic.

Exemplary non-proteinaceous moieties which may be used according to the present teachings include, but are not limited to, polyethylene glycol (PEG), Polyvinyl pyrrolidone (PVP), poly(styrene comaleic anhydride) (SMA), and divinyl ether and maleic anhydride copolymer (DIVEMA).

Such a molecule is highly stable (resistant to in-vivo proteolytic activity probably due to steric hindrance conferred by the non-proteinaceous moiety) and may be produced using common solid phase synthesis methods which are inexpensive and highly efficient, as further described herein below. However, it will be appreciated that recombinant techniques may still be used, whereby the recombinant polypeptide product is subjected to in-vitro modification (e.g., PEGylation as further described herein below).

Bioconjugation of non-proteinaceous moieties (such as PEGylation) can confer the isolated polypeptide's with stability (e.g., against protease activities) and/or solubility (e.g., within a biological fluid such as blood, digestive fluid) while preserving its biological activity and prolonging its half-life.

Bioconjugation is advantageous particularly in cases of therapeutic proteins which exhibit short half-life and rapid clearance from the blood. The increased half-lives of bioconjugated proteins in the plasma results from increased size of protein conjugates (which limits their glomerular filtration) and decreased proteolysis due to polymer steric hindrance. Generally, the more polymer chains attached per polypeptide, the greater the extension of half-life. However, measures are taken not to reduce the specific activity of the isolated polypeptide or fusion protein of the present invention (e.g. capability of binding ACE2).

Bioconjugation of the isolated polypeptide's amino acid sequence with PEG (i.e., PEGylation) can be effected using PEG derivatives such as N-hydroxysuccinimide (NHS) esters of PEG carboxylic acids, monomethoxyPEG₂-NHS, succinimidyl ester of carboxymethylated PEG (SCM-PEG), benzotriazole carbonate derivatives of PEG, glycidyl ethers of PEG, PEG p-nitrophenyl carbonates (PEG-NPC, such as methoxy PEG-NPC), PEG aldehydes, PEG-orthopyridyl-disulfide, carbonyldimidazol-activated PEGs, PEG-thiol, PEG-maleimide. Such PEG derivatives are commercially available at various molecular weights [See, e.g., Catalog, Polyethylene Glycol and Derivatives, 2000 (Shearwater Polymers, Inc., Huntsvlle, Ala.)]. If desired, many of the above derivatives are available in a monofunctional monomethoxyPEG (mPEG) form.

In general, the PEG added to the isolated polypeptide's amino acid sequence of the present invention should range from a molecular weight (MW) of several hundred Daltons to about 100 kDa (e.g., between 3-30 kDa). Larger MW PEG may be used, but may result in some loss of yield of PEGylated peptides. The purity of larger PEG molecules should be also watched, as it may be difficult to obtain larger MW PEG of purity as high as that obtainable for lower MW PEG. It is preferable to use PEG of at least 85% purity, and more preferably of at least 90% purity, 95% purity, or higher PEGylation of molecules is further discussed in, e.g., Hermanson, Bioconjugate Techniques, Academic Press San Diego, Calif (1996), at Chapter 15 and in Zalipsky et al., “Succinimidyl Carbonates of Polyethylene Glycol,” in Dunn and Ottenbrite, eds., Polymeric Drugs and Drug Delivery Systems, American Chemical Society, Washington, D.C. (1991).

Conveniently, PEG can be attached to a chosen position in the isolated polypeptide's amino acid sequence by site-specific mutagenesis as long as the activity of the conjugate is retained (e.g. capability of binding ACE2). A target for PEGylation could be any Cysteine residue at the N-terminus or the C-terminus of the isolated polypeptide's amino acid sequence. Additionally or alternatively, other Cysteine residues can be added to the isolated polypeptide's amino acid sequence (e.g., at the N-terminus or the C-terminus) to thereby serve as a target for PEGylation. Computational analysis may be effected to select a preferred position for mutagenesis without compromising the activity.

Various conjugation chemistries of activated PEG such as PEG-maleimide, PEG-vinylsulfone (VS), PEG-acrylate (AC), PEG-orthopyridyl disulfide can be employed. Methods of preparing activated PEG molecules are known in the arts. For example, PEG-VS can be prepared under argon by reacting a dichloromethane (DCM) solution of the PEG-OH with NaH and then with di-vinylsulfone (molar ratios: OH 1: NaH 5: divinyl sulfone 50, at 0.2 gram PEG/mL DCM). PEG-AC is made under argon by reacting a DCM solution of the PEG-OH with acryloyl chloride and triethylamine (molar ratios: OH 1: acryloyl chloride 1.5: triethylamine 2, at 0.2 gram PEG/mL DCM). Such chemical groups can be attached to linearized, 2-arm, 4-arm, or 8-arm PEG molecules.

While conjugation to cysteine residues is one convenient method by which the isolated polypeptide's amino acid of the present invention can be PEGylated, other residues can also be used if desired. For example, acetic anhydride can be used to react with NH₂ and SH groups, but not COOH, S—S, or —SCH₃ groups, while hydrogen peroxide can be used to react with —SH and —SCH₃ groups, but not NH₂. Reactions can be conducted under conditions appropriate for conjugation to a desired residue in the polypeptide employing chemistries exploiting well-established reactivities.

For bioconjugation of the isolated polypeptide's amino acid sequence of the present invention with PVP, the terminal COOH-bearing PVP is synthesized from N-vinyl-2-pyrrolidone by radical polymerization in dimethyl formamide with the aid of 4,4′-azobis-(4-cyanovaleric acid) as a radical initiator, and 3-mercaptopropionic acid as a chain transfer agent. Resultant PVPs with an average molecular weight of Mr 6,000 can be separated and purified by high-performance liquid chromatography and the terminal COOH group of synthetic PVP is activated by the N-hydroxysuccinimide/dicyclohexyl carbodiimide method. The isolated polypeptide's or fusion protein's amino acid sequence is reacted with a 60-fold molar excess of activated PVP and the reaction is stopped with amino caploic acid (5-fold molar excess against activated PVP), essentially as described in Haruhiko Kamada, et al., 2000, Cancer Research 60: 6416-6420, which is fully incorporated herein by reference.

Resultant conjugated isolated polypeptide (e.g., PEGylated or PVP-conjugated isolated polypeptide) are separated, purified and qualified using e.g., high-performance liquid chromatography (HPLC). In addition, purified conjugated molecules of this aspect of the present invention may be further qualified using e.g., in vitro assays in which the binding specificity of the isolated peptide to its ligand (e.g., lysyl oxidase) is tested in the presence or absence of the isolated polypeptide, essentially as described for other polypeptides e.g. by surface plasmon resonance assay or by yeast display assay.

Molecules of this aspect of present invention can be biochemically synthesized such as by using standard solid phase techniques. These methods include exclusive solid phase synthesis, partial solid phase synthesis methods, fragment condensation and classical solution synthesis. These methods are preferably used when the polypeptide is relatively short (i.e., 10 kDa) and/or when it cannot be produced by recombinant techniques (i.e., not encoded by a nucleic acid sequence) and therefore involve different chemistry.

Thus, the polypeptides of some embodiments of the invention may be synthesized by any techniques that are known to those skilled in the art of polypeptide synthesis. For solid phase polypeptide synthesis, a summary of the many techniques may be found in J. M. Stewart and J. D. Young, Solid Phase Polypeptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing polypeptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage.

The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final polypeptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of polypeptide synthesis is disclosed in U.S. Pat. No. 6,472,505.

A particular method of preparing the polypeptide compounds of some embodiments of the invention involves solid phase polypeptide synthesis.

Large scale polypeptide synthesis is described by Andersson Biopolymers 2000; 55(3):227-50.

Synthetic polypeptides can be purified by preparative high performance liquid chromatography [Creighton T. (1983) Proteins, structures and molecular principles. W H Freeman and Co. N.Y] and the composition of which can be confirmed via amino acid sequencing.

In cases where large amounts of the polypeptides of the present invention are desired, the polypeptides of the present invention can be generated using recombinant techniques such as described by Bitter et al. (1987) Methods in Enzymol. 153:516-544; Studier et al. (1990) Methods in Enzymol. 185:60-89; Brisson et al. (1984) Nature 310:511-514; Takamatsu et al. (1987) EMBO J. 6:307-311; Coruzzi et al. (1984) EMBO J. 3:1671-1680; Brogli et al. (1984) Science 224:838-843; Gurley et al. (1986) Mol. Cell. Biol. 6:559-565 and Weissbach & Weissbach, 1988, Methods for Plant Molecular Biology, Academic Press, NY, Section VIII, pp 421-463.

For example, a nucleic acid sequence encoding the peptide of the present invention is ligated to a nucleic acid sequence which may include an inframe sequence encoding a proteinaceous moiety such as immunoglobulin.

Also provided is an expression vector, comprising the isolated polynucleotide of some embodiments of the invention. According to one embodiment, the polynucleotide sequence is operably linked to a cis-acting regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion from a host cell in which it is placed. Preferably the signal sequence for this purpose is a mammalian signal sequence.

Preferably, the promoter utilized by the nucleic acid construct of some embodiments of the invention is active in the specific cell population transformed.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Also provided are cells which comprise the polynucleotides/expression vectors as described herein.

Suitable host cells for cloning or expression include prokaryotic or eukaryotic cells. See e.g. Charlton, Methods in Molecular Biology, Vol. 248 (B. K. C. Lo, ed., Humana Press, Totowa, N. J., 2003), pp. 245-254, describing expression of antibody fragments in E. coli; see Gerngross, Nat. Biotech. 22:1409-1414 (2004), and Li et al., Nat. Biotech. 24:210-215 (2006) for suitable fungi and yeast strains; and see e.g., U.S. Pat. Nos. 5,959,177, 6,040,498, 6,420,548, 7,125,978, and 6,417,429 for suitable plant cell cultures which can also be utilized as hosts.

After expression, the isolated polypeptide may be isolated from the cells in a soluble fraction and can be further purified.

Recovery of the isolated polypeptide may be effected following an appropriate time in culture. The phrase “recovering the recombinant polypeptide or fusion protein” refers to collecting the whole fermentation medium containing the polypeptide or fusion protein and need not imply additional steps of separation or purification.

Notwithstanding the above, proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Molecules of the present invention are preferably retrieved in “substantially pure” form. As used herein, “substantially pure” refers to a purity that allows for the effective use of the protein in the applications, described herein.

The peptide may be used per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the peptide accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include topical, oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping, spray drying, coating or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (peptide) effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Mice maintenance and genotyping: All mice are housed in IVC's (Techniplast) according to space requirements defined by the NRC. All rooms are set to have 22±2° C. and humidity of 30-70%. HVAC parameters and light cycle is set by the computerized central system to full light of 10 hours, half-light of 2 hours and complete darkness of 12 hours. All mice were bred on a C57Bl/6 background purchased from Envigo (https://www(dot)envigo(dot)com). Embryonic day was staged according to Kaufmann, whereby the morning of the day in which a vaginal plug was observed was marked as E0.5. Mice were genotyped using PCR and the following primers: LoxL3: 5′-GCCAGGGTGAAGTGAAAGAC-3′ (SEQ ID NO: 1); 3′-GATCTGGGATGCTGAAGACC-5′ (SEQ ID NO: 2); 3′-GAACTTCGGAATAGGAACTTCG-5′ (SEQ ID NO: 3). 300 bp and 100 bp represent wild-type and mutant PCR products, respectively.

Embryos' dissection, imaging and analysis: E9.5 and E18.5 embryos were dissected and underwent over-night fixation after somites were counted. Afterwards, whole mount staining was performed for the E9.5 embryos using FN and ap2α antibodies. Neural crest nuclei in the second branchial arch were then counted from confocal images. For analyses of FN organization in the somite borders, z-stack images were taken. The trainable Weka segmentation plugin (41) of ImageJ (NIH) software was used to identify FN fibrils in each stack. The area of the quantified fibrils was divided by the total somite area in each stack and the ratios were then averaged. For detection of cleft palate, E18.5 embryos underwent alcian blue and alizarin red staining.

Wholemount embryo staining: Embryos were incubated in 600 μl of blocking buffer (PBS, 0.01% Triton X-100 and 10% non-immune donkey serum (Sigma cat #D9663-10 ml) overnight at 4° C., then with 600 μl of blocking buffer containing primary antibodies for 4 days at 4° C., with gentle rocking. The following primary antibodies were used: anti-Fn1 (Abcam cat #199056, 1:500), anti-TFAP2α (Developmental Studies Hybridoma Bank, DSHB-3B5, 1:200). After the incubation with primary antibodies, embryos were rinsed and washed with PBST (PBS with 0.05% Triton X-100) for 2 days at 4° C., with gentle rocking. Embryos were then incubated with 600 μl of blocking buffer containing DAPI (ThermoFisher, cat #D3571, 5 mg/ml stock diluted 1:1000) and secondary antibodies diluted 1:300 for 4 days at 4° C. Alexa-labeled secondary antibodies were purchased from Invitrogen (donkey anti-mouse Alexa-647 cat #A31571 and donkey anti-rabbit Alexa-488 cat #A21206). After staining with DAPI and secondary antibodies, embryos were washed with PBST. Prior to imaging, embryos were dehydrated gradually with 25%, 50%, 75% of methanol diluted in dH2O for 10 minutes for each step, and then incubated with 100% methanol twice for 10 min each, and cleared in 50% BABB (BABB and 100% methanol at 1:1 (v/v)), and 100% BABB. BABB was generated by mixing benzyl alcohol (Sigma, #B1042) and benzyl benzoate (Sigma, #B6630) at 1:2 (v/v). Cleared embryos were placed between two coverslips (VWR cat #16004-312) separated by FastWells spacers (Grace Bio-Labs, cat #664113). Images were acquired using Nikon A1-HD25 inverted microscope equipped with a 20× water immersion objective (cat #MRD77200), numerical aperture 0.95, working distance 0.95 mm, and the NIS-Elements AR 5.11.01 64-bit software. Optical sections were collected at 0.55 μm intervals.

Cell culture: MEF FN-GFP were described previously (24). MEF Paxillin-GFP cells were kindly provided by B. Geiger (Weizmann institute of Science, Israel). Dermal fibroblasts were kindly provided by R. Shalom-Feuerstein (Technion, Israel). The three cell lines were cultured in DMEM (Sigma) with 10% FBS, 1% glutamine and 1% pen-strep (Biological Industries). HASMC (Sigma, 354-05A) were cultured with Smooth Muscle Cell Growth Medium 2 (PromoCell, C-22082).

Generation of mutant FN constructs: Point mutations were generated on a human fibronectin pMAX vector plasmid (Addgene, #120402) using the Q5 site-directed mutagenesis kit (BioLabs, #E05545). The mutagenesis procedure was performed using the following primers: Oxidation site 1: 5′-TGAGCGTCCTGCAGACTCCATG-3′ (SEQ ID NO: 4), 3′-TAAGTGTCACCCACTCGG-5′ (SEQ ID NO: 5); RGD: 5′-GCCGTGGAGAAAGCCCCGCAA-3′ (SEQ ID NO: 6), 3′-CAGTGACAGCATACACAGTGATGGTATAATCAAC-5′ (SEQ ID NO: 7); Oxidation site 2: 5′-TGATGATGGGGCGACATACCACG-3′ (SEQ ID NO: 8), 3′-TAACACGTTGCCTCATGAG-5′ (SEQ ID NO: 9); Oxidation site 3: 5′-ACAGTGGCAGGCGGAATATCTCG-3′ (SEQ ID NO: 10), 3′-TCTCCTACGTGGTATGTC-5′ (SEQ ID NO: 11). Next, mutated FN constructs were PCR amplified and ligated into BamH1+Xhol cut NSPI vector using NEBuilder® HiFi DNA Assembly kit (BioLabs, #E5520).

Lentiviral infection: Plasmids were transfected into HEK 293-FT cells using CalFectin™ Mammalian Cell Transfection Reagent (SignaGen, SL100478). After 48 hours, conditioned medium was collected and used to infect MEF or HASMC cells. Plasmids used: Fibronectin mutant constructs, shLOX (Sigma, TRCN0000045991), shControl (Sigma, SHC016).

Spreading and soft gel experiments: Ibidi 8-wellplates (Ibidi, 190814/1) were coated with human plasma FN (hFN, Sigma-Aldrich, FC010-10MG, 10 μg/ml) for 1 hour at 37° C. Afterwards, the samples were treated with human recombinant LOXL3 (RhLOXL3, R&D systems, 6069-AO) or LOXL2 (RhLOXL2, R&D systems, 2639-AO) for 5 hours (or sodium borate buffer 50 mM as control), with or without 200 mM βAPN (Sigma, A3134-25G) and then washed. Then, cells were seeded on the FN matrix and fixed at varying time points after spreading, followed by immunostaining. For soft gel experiments, Ibidi 8-well plates were covered with silicone gels (DOWSIL, CY 52-276 A&B), prepared at different rigidities that were previously calibrated and described (42) previous to FN coating. Images were acquired using an LSM800 confocal microscope (Zeiss) with Airyscan function and 20× or 63× objectives.

Adhesion, cell area andfibronectin analysis: All of the adhesion and fibronectin fibrils analyses shown in this paper were performed using the trainable Weka segmentation plugin (41) of ImageJ (NIH) software.

Immunofluorescence: Cells were fixed with 4% paraformaldehyde solution (36% stock, Sigma-Aldrich, 50-00-0, diluted with PBS) and permeabilized with 0.1% Triton X-100 in PBS (PBT 0.1%). Primary antibodies were incubated with the fixed cells over night at 4° C. After, secondary antibodies and phalloidin were incubated for 1 hour at room temperature. For dSTORM imaging, a second 10 minutes fixation was performed at the end of the immunostaining protocol.

Antibodies that were used: anti-total Paxillin (Abcam, ab32084, 1:200); anti-FN (Abcam, ab2413, 1:500); anti-LoxL3 and anti-LoxL2 (produced and kindly gifted by G. Neufeld, Technion). Phalloidin (Abcam, ab176757, 1:1000) and DAPI (Biolegend, 422801, 1:10,000) were also used.

Single cell migration assay: FN was treated with RhLOXL3, with or without βAPN as described above. Then, 24-well cell imaging plates (Eppendorf, EP-0030741005) were coated with the treated or untreated FN. Cells were incubated with 200 nM Sir-DNA (SpiroChrome, SC007) for 6 hours, and were then seeded. Two hours after seeding, a series of 64 images was taken (4 images/h) using the ImageXpress Micro Confocal High-Content Imaging system (Molecular Devices). Nuclei tracking analysis was performed using IMARIS software (Oxford instruments).

dSTORM imaging and analysis: Prior to imaging, FN was treated with RhLOXL3 as described above (molar ratio of 25:1) for 5 hours and was then diluted to 1 ng/ml. Then, #1.5 coverslips were coated with the diluted FN (treated, untreated or fresh FN without any incubation). To detect FN, aFN (Abcam, ab2413) and Alexa-647 conjugated secondary antibodies were used. Fresh dSTORM buffer was prepared as previously described (43). 2D dSTORM images were taken using a SAFe360 module (Abbelight Ltd, Cachan, France) coupled to Olympus IX83 inverted microscope using a 100× oil-immersion TIRF objective (NA 1.49) and 640 nm laser. The system is equipped with two sCMOS cameras PCO.panda4.2. A total of 15,000 frames at 50 ms exposure time were acquired and used for single-molecule detections to reconstruct a dSTORM image. Resulting coordinate tables and images were processed and analyzed using SAFe NEO software (Abbelight Ltd, Cachan, France).

3D dSTORM images were acquired in a similar manner with the following changes: LoxL3 was detected using anti-LoxL3 antibody. F-actin was detected using Phalloidin-647 (Abcam abl76759). For identification of the Z axis position of each single molecule an astigmatic lens was place in front of the cameras. 40 nm fluorescent beads were added to the sample prior to cell seeding in order to correct the x-y-z drifts.

Pillar array preparation and imaging: Pillar fabrication was performed by pouring Polydimethylsiloxane (PDMS) (mixed at 10:1 with its curing agent, Sylgard 184; Dow Corning) into silicon molds (fabricated as previously described (32)) with holes at fixed depths and distances (center-to-center distance=1 μm). The molds were then placed, face down, onto glass-bottom 35 mm dishes (#0 coverslip, Cellvis) which were incubated at 65° C. for 12 h to cure the PDMS. The molds were next peeled off while immersed in ethanol to prevent pillar collapse. On the day of the experiment, FN was treated with RhLOXL3 for 5 hours (or sodium borate buffer 50 mM as control). Then, the pillars were coated with the treated or untreated FN for 1 hour at 37° c. Next, the buffer was replaced to HBSS+HEPES 20 mM (Biological industries), pH 7.2.

Pillar bending stiffness (k) was calculated by Euler-Bernoulli beam theory as described:

$$k = \frac{3 π}{64} \frac{E {D}^{4}}{{L}^{3}}$$

where D and L are the diameter and length o the pillar, respectively, and E is the Young's modulus of the PDMS(=2 MPa).

Cells were resuspended with the HBSS/HEPES buffer and then spread on the FN-coated pillars. Time-lapse imaging of cells spreading on the pillars was performed using an inverted microscope (Leica DMIRE2) at 37° C. using a 63×1.4 NA oil immersion objective. Brightfield images were recorded every 2 seconds with a Retiga EXi Fast 1394 CCD camera (QImaging). The microscope and camera were controlled by Micromanager software (44). For each cell, a movie of 10-20 minutes was recorded. To minimize photo-damage to the cells, a 600 nm longpass filter was inserted into the illumination path.

Pillar displacement analysis: Pillar tracking was performed using the Nanotracking plugin of ImageJ, as described previously (30). In short, the cross-correlation between the pillar image in every frame of the movie and an image of the same pillar from the first frame of the movie was calculated, and the relative x- and y-position of the pillar in every frame of the movie was obtained. To consider only movements of pillar from their zero-position, the present inventors only analyzed pillars that at the start of the movie were not in contact with the cell and that during the movie the cell edge reached to them. Drift correction was performed using data from pillars far from any cell in each movie. For each pillar, the displacement curve was generated by Matlab (MathWorks). Analyses of the catch-and-release events was performed by the ‘findpeaks’ function in Matlab, considering all the peaks above noise level with a minimum width of 3 frames.

Statistical analysis and plotting: Matlab was used for statistical analysis and graph plotting. For all of the boxplots shown in the paper, the red central mark represents the median, the bottom and the top edges of the box represents the 25th and 75th percentiles, respectively, and the black top and bottom represent the minimal and maximal values that are not outliers, respectively. Outliers are represented as red crosses.

Western Blot: Protein lysates were harvested from the cell lines using a lysis buffer (Tris 10 mM pH7, 2 nM EDTA, 1% NP-40, 0.1% DOC, 0.2 mM AEBSF). The lysates were loaded onto SDS-PAGE. Gels were transferred to nitrocellulose membrane that was blocked for 1 hour (5% BSA, 0.1% Tween in TBS). The membranes were then probed with the primary antibody over night at 4° C. Secondary antibody was added for 2 hours at room temperature. Antibodies used for this method: anti-Lox (58135, Cell Signaling); anti-P97 (kindly provided by A. Stanhill, Technion); anti-Fibronectin (Abcam, ab23750).

Neural crest explants: Neural tube explants were carried out essentially as previously described (45). Briefly, hindbrain regions of E8-8.5 embryos were dissected and the neural primordia consisting of the pre-migratory neural crest cells was isolated from surrounding tissues using fine dissecting pins. The neural tubes were then cultured on FN (50 μg/ml) coated dishes and incubated for 16 hours at 37° C. in DMEM supplemented with 10% fetal bovine serum, 1% 1M HEPES, 1% PenStrep, and 1% L-glutamate (Gibco, USA). Explants were then fixed with 4% PFA and immunofluorescently stained.

### Results

### Lysyl Oxidases Promote FN Fibrillogenesis and Cell Spreading on Soft Matrices

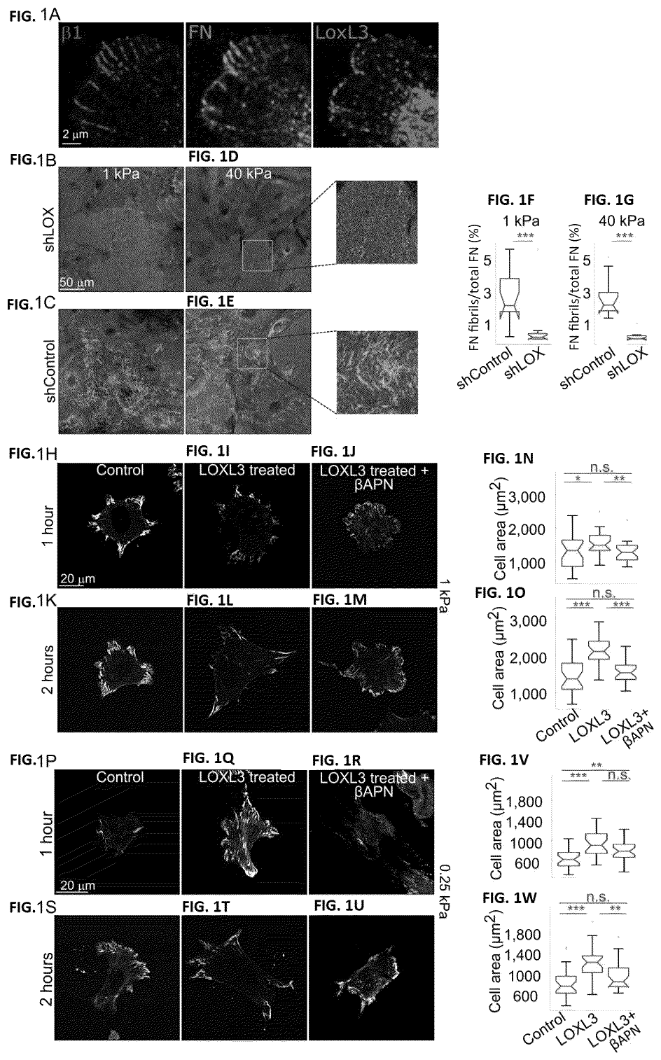

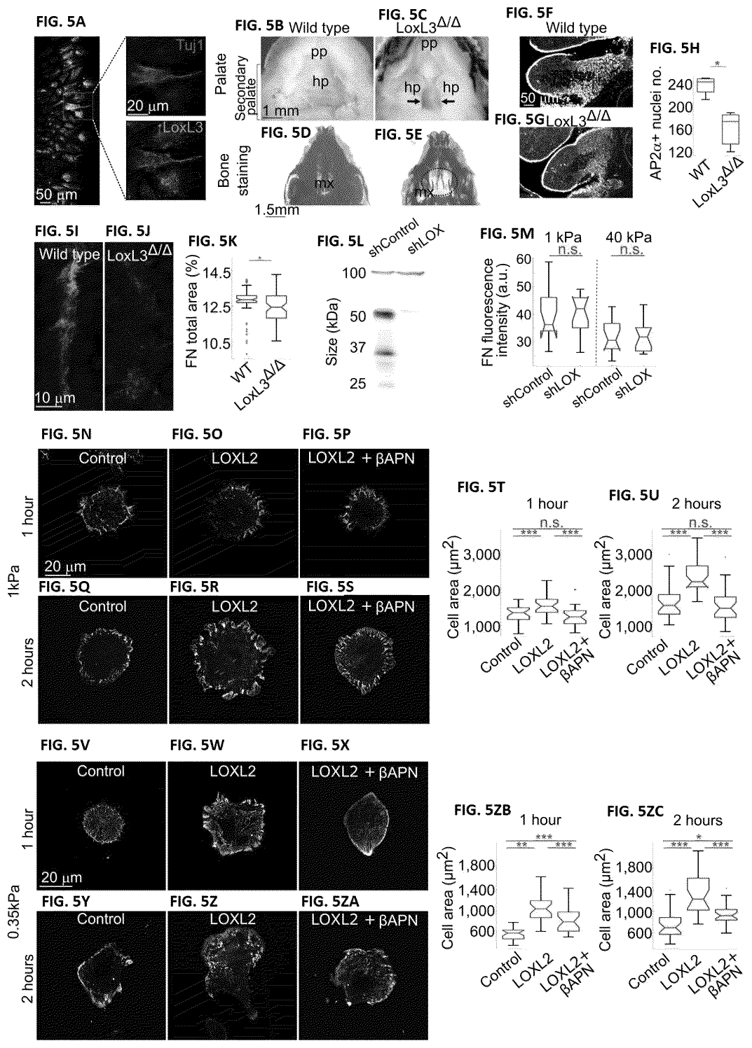

The finding that members of the Lysyl oxidase enzyme family bind FN, and that LOX-Like 3 (LOXL3) oxidizes it (23), raised the possibility of an enzyme-dependent initiation of fibrillogenesis, a force-independent mechanism which relies on enzymes and their substrates' availability. In line with this notion, in cultured cells expressing GFP-tagged FN (24), LoxL3 is distributed at nascent adhesion sites in regions where FN is present but not yet at the fibrillar form, whereas FN fibrils appear primarily under mature R 1-integrin adhesions (FIG. 1A). Notably, LoxL3 is expressed in neural crest cells (FIG. 5A) and following its deletion (LoxL3^{Δ/Δ}) the mutant embryos display FN-associated phenotypes such as cleft palate (25) and reduced neural crest cell numbers at branchial arches (26) (FIG. 5B-H). Further, abnormal FN-fibril formation along the somitic boundaries is also observed in LoxL3^{Δ/Δ} mutant embryos (FIG. 5I-K).

Collectively, these findings suggest a role for Lysyl oxidases in FN fibrillogenesis. To test the relationship between substrate rigidity, FN fibrillogenesis, and the Lysyl oxidases, the present inventors took advantage of primary human aortic smooth muscle cells (HASMCs), which a) normally reside on a rigid artery wall and, depending on stretching, their direct rigidity is 15-88 kPa (27); and b) primarily express the lysyl oxidase family member, LOX (28). Following infection with short hairpin RNA against LOX (shLOX) or scrambled RNA (shCtrl) (FIG. 5L), the cells were cultured for 24 hrs on 1 kPa or 40 kPa FN-coated surfaces and monitored FN-fibrils by immunostaining. Whereas shCtrl cells displayed significant FN fibril formation, cells devoid of LOX, despite secreting FN to the same extent (FIG. 5M), did not form any FN fibrils on both rigidities (FIG. 1B-G).

The converse experiment was then carried out in order to test whether pre-treatment of FN with lysyl oxidases overrides rigidity sensing and promotes early cell adhesion on soft matrices. Towards that end, recombinant human LOXL3 or LOXL2, was used and FN-coated 1.0 and 0.25 kPa surfaces were treated with either enzyme. Mouse embryonic fibroblasts (MEF) stably expressing Paxillin-GFP (Pax-GFP) were then added onto the plates and fixed after 60 and 120 minutes on the sham-, LOXL3- or LOXL2-treated FN (FIGS. 1H-W and FIGS. 5N-ZC). FN fibrils were undetectable at these early times of cell spreading and therefore cell areas were monitored as readouts of cellular interaction with FN. This analysis revealed a significant increase in the spreading of cells plated on the LOXL3- or LOXL2-treated FN in comparison to the sham-treated FN or to those treated with the enzyme mixed with the pan LOX inhibitor beta-aminopropionitrile (βAPN) (29). Altogether, these results suggest a general role for the enzymatic activity of lysyl oxidases that promotes FN fibrillogenesis and cell adhesion.

### LOX-Treated FN Promotes Nascent Adhesion Formation

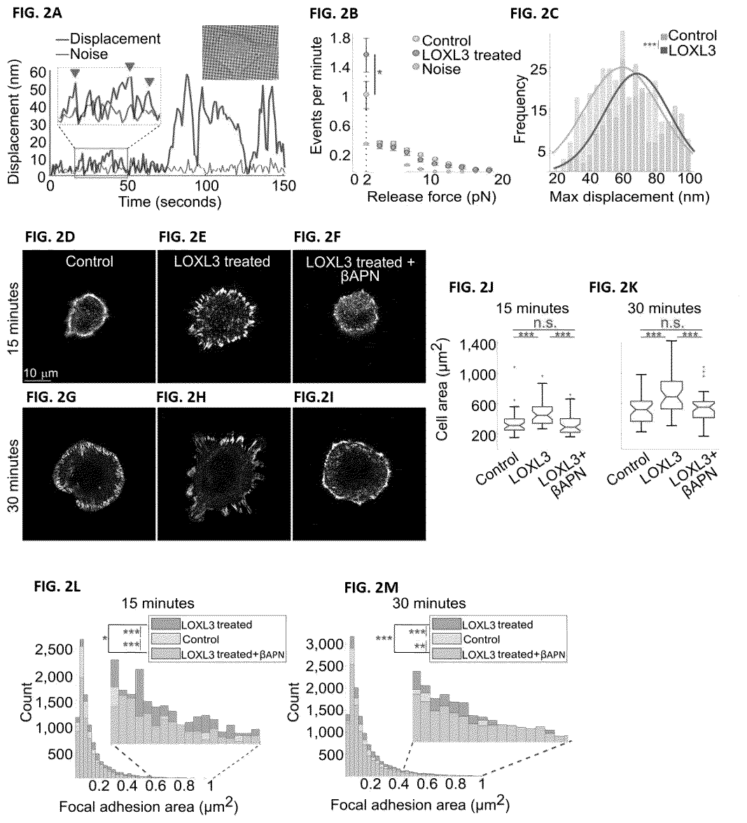

The above results suggest that LOX family members modulate the cells' response to the external rigidity by initiating FN fibrillogenesis and enhancing adhesion formation. Direct treatment of FN with a lysyl oxidase was analyzed to see if it affected the mechanism by which cells perform mechanosensing of ECM rigidity. It was postulated that the enzymatic activity of lysyl oxidases promotes a structural change in FN that allows creating more stable nascent adhesions through which early mechanosensing of matrix rigidity occurs (30, 31). To test this, cells were plated on arrays of deformable PDMS pillars, with 0.5 μm diameter and 2.3 μm height (bending stiffness 1.5 pN/nm (30, 32), equivalent to ˜2 kPa (33)), and live-cell time-lapse imaging of early attachment and spreading of the cells was performed (FIG. 2A-C). To test the hypothesis that LOX-induced local structural change in FN enhances nascent adhesion formation, the pillars were coated with FN at a density which was less than saturation level (1 μg/ml, equivalent to ˜25% coverage of the pillar tops). Analyses of early times of cell edge interactions with individual pillars revealed repeated catch-and-release events above noise level (FIG. 2A). The strength of the linkage in each of these events was analyzed by measuring the pillar displacement level at the time of release, translating it to force. Comparison of the histograms of release forces between sham- and LOXL3-treated FN showed that the latter displayed significantly more events per unit time at 2 pN (FIG. 2B), consistent with the formation of integrin-mediated FN-actin 2 pN bonds in the presence of trimers of FN's integrin-binding domain FNIII7-10, but not with monomers (34). These results are suggestive of formation of more closely packed FN clusters following LOXL3 treatment. Further, analyses of the longer-term pillar displacements that succeeded the initial catch-and-release events showed a significant shift toward higher values of the maximal displacement histogram in LOXL3-treated compared to control FN (FIG. 2C). Together, these results suggest that the ability to form nascent adhesions on soft surfaces is enhanced upon LOX treatment, which assists in overcoming the low rigidity of the matrix.

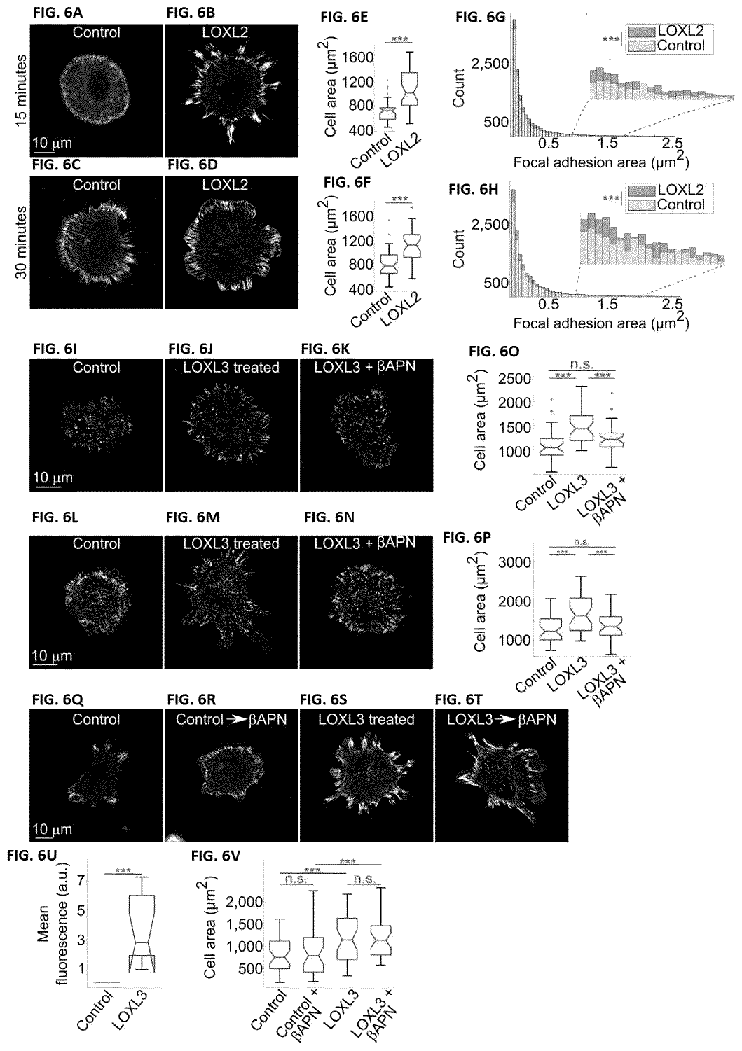

Nascent adhesion formation, even on very stiff matrices, requires at least four RGD (Arg-Gly-Asp) ligands that are localized within ˜60 nm from each other (35, 36). Thus, if LOX treatment led to an adhesion promoting structural change in FN, one should expect to see a difference in early adhesion even on stiff surfaces such as glass whose stiffness is at the GPa range. Towards that end, following enzymatic treatment, FN was coated on glass coverslips and Pax-GFP cells were added and shortly after (15 or 30 minutes) fixed and analyzed. Quantifications of adhesion sizes at 15 minutes showed a significant increase in the relative count of adhesions larger than 0.1 μm² on LOXL3- or LOXL2-treated FN and to a concomitant increase in cell spreading area (FIGS. 2D-F, J, L and FIGS. 6A-B, E, G).

Importantly, the increases in adhesion sizes and cell areas were blocked when βAPN was added to the FN-LOXL3 mix throughout the reaction (FIGS. 2J, L). A similar trend was observed at 30 minutes, albeit to a lesser extent (FIGS. 2G-L, K, M and FIGS. 6C-D, F, H), altogether suggesting that the primary effect of the enzymatic treatment occurred at the very early stages of adhesion assembly. A comparable effect was observed with two other fibroblast cells (FIG. 6I-P), indicating that the effect of lysyl oxidases on adhesion to FN was not specific to a particular cell line. Notably, since α5β1 integrin can interact with LOX enzymes (37), it was verified that the observed effect on adhesion occurred through changes in FN and not through direct integrin activation by residual enzyme that remained attached to FN (FIG. 6U). Indeed, when βAPN was added following the FN treatment, no difference was observed in cell spreading compared to treated FN without the inhibitor (FIGS. 6Q-T, V), demonstrating that the enzymatic activity of LOXL3 directly affects FN and that the presence of the enzyme by itself (when inactive) has no effect. Taken together, the similar results observed following LOXL2 and LOXL3 treatment (FIGS. 2A-M and FIGS. 6A-V), and the observations on LOX effects on FN fibrillogenesis (FIGS. 1A-W and FIGS. 5A-5ZC), reinforce the idea of a general enzymatic role among LOX family members in affecting cell adhesion through initiation of FN fibrillogenesis.

### LOX Induces FN Clustering

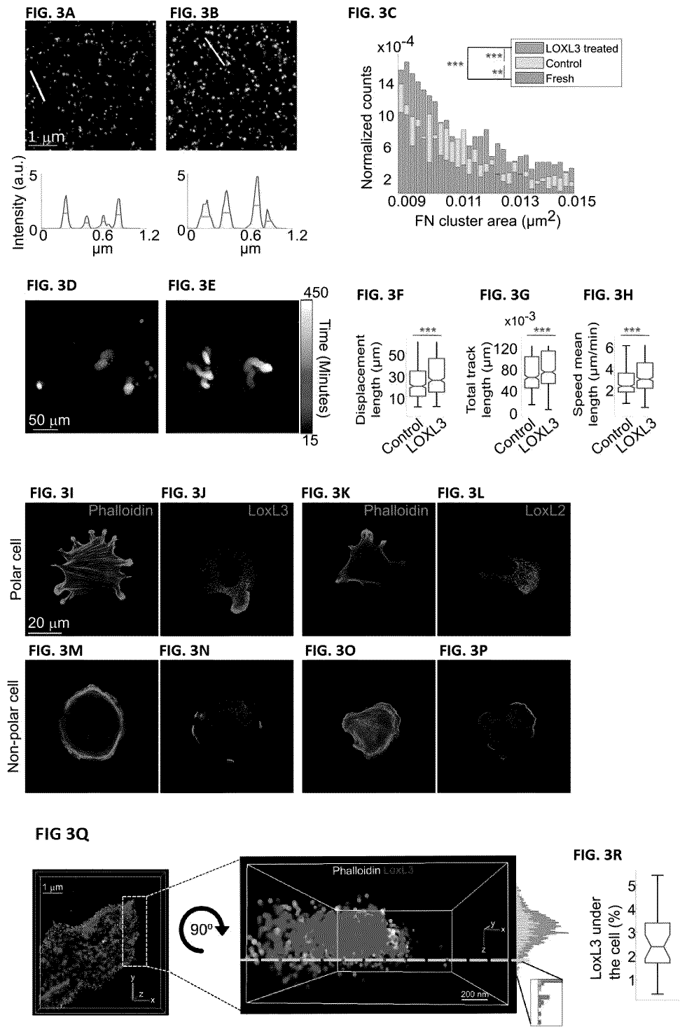

Next, the present inventors directly tested whether alteration to FN organization occurs following LOX treatments. To this end, they deposited treated and untreated FN samples, as well as fresh FN, on glass coverslips at low density (100 ng/ml). After immunostaining the samples, they imaged regions completely devoid of cells using direct stochastic optical reconstruction microscopy (dSTORM). Particle analysis was used to identify FN clusters in the dSTORM images (FIG. 3A-B). The histograms of the detected FN clusters indicate that LOXL3 treatment led to significant increase in the number of clusters larger than 0.009 μm² compared to fresh or sham-treated FN (FIG. 3C). Collectively, these results indicate that LOX treatment enhances FN clustering, which results in cells perceiving the matrix as stiffer.

Since adhesion stabilization plays a major role in maintenance of a leading edge in cell migration (38), it was postulated that the larger FN clusters following treatment would lead to improved migration. Pax-GFP cells were plated on sham- and LOXL3-treated FN and live-cell imaging was performed to track their migratory patterns and quantify the overall displacement length, migration distance, and speed (FIG. 3D-H). All three parameters were significantly increased following LOXL3 FN-treatment, demonstrating that not only were the cells able to rapidly generate adhesions on the treated FN but, importantly, that the cells' migration pattern was more persistent (evident by the increased displacement length), further reinforcing the notion that the adhesions that form on the LOXL3-treated FN are more stable.

The overall results thus far suggest that exogenous FN treatment with a member of the lysyl oxidase family promotes a faster and more robust cell adhesion. As members of the family are secreted enzymes, their secretion was analyzed in order to determine whether it was localized to cellular regions where such robust integrin-dependent adhesions are mostly required. To that end, Pax-GFP cells were immunostained for LoxL2 or LoxL3 following 60 minutes of spreading. It was found that in polarized cells, LoxL2 and LoxL3 expression is typically localized to the most prominent lamellipodial protrusion of the cell (FIG. 3I-L). Surprisingly, even in non-polarized cells, narrow stripes in lamellipodial regions were also observed (FIG. 3M-P). Further, 3-dimensional (3D) dSTORM analysis demonstrates that these matrix modifying enzymes are specifically secreted from the leading edge (FIG. 3Q-R). Taken together, these results support a model whereby localized secretion of LOX enzymes at the cell edge enhances FN clustering, thereby supporting nascent adhesion formation and persistent cell migration.

### FN Oxidation is Essential for FN Fibrillogenesis

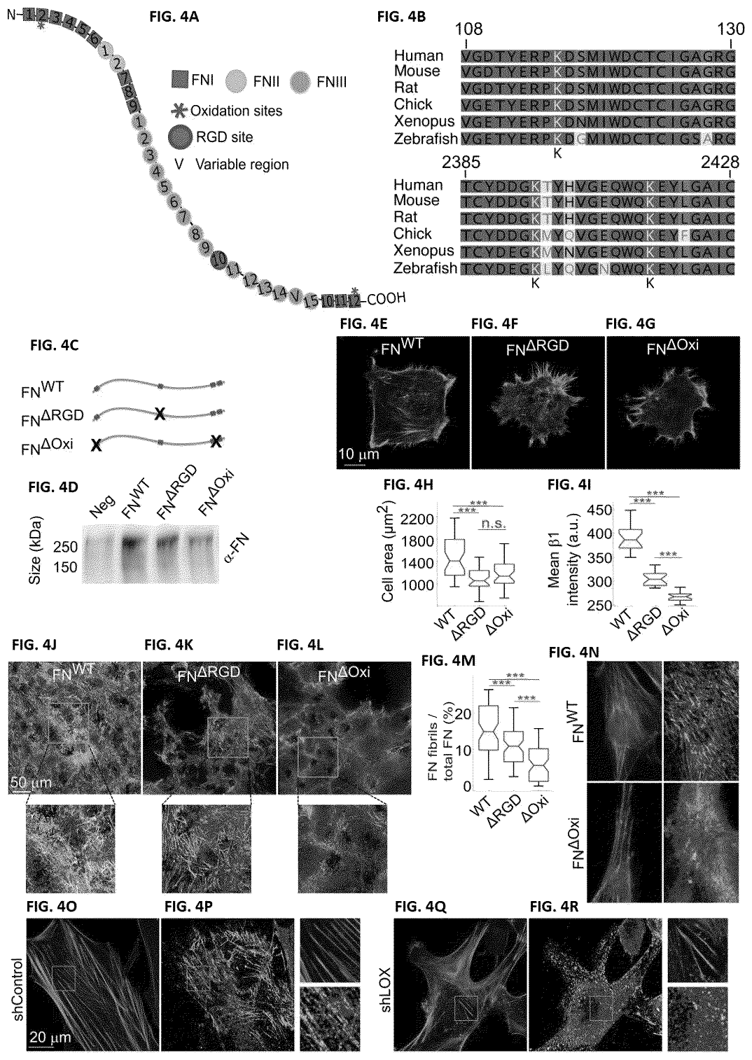

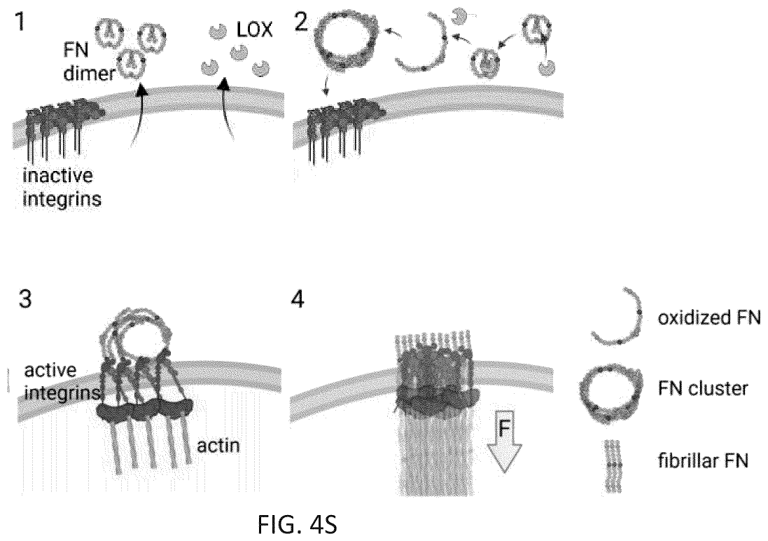

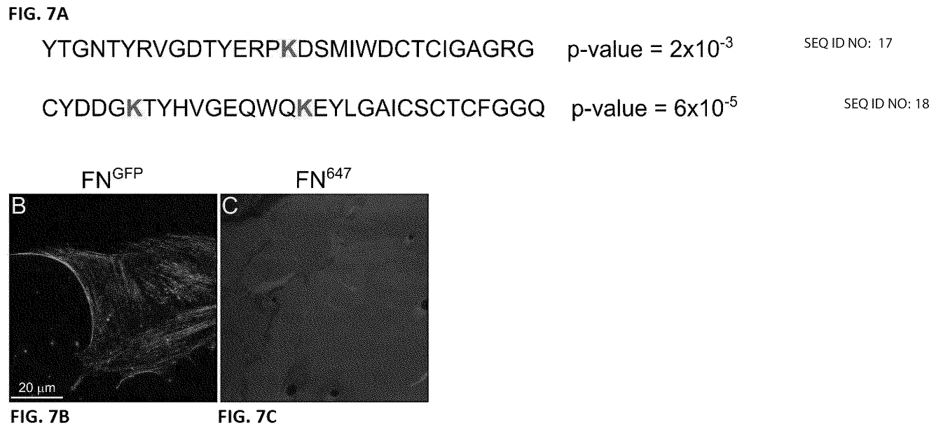

Since a) Lysyl oxidases' enzymatic activity is required for FN-mediated cell adhesion and migration; b) treatment of FN with LOXL3 promotes its clustering; and c) LOXL3 oxidizes FN (23), the present inventors further explored the oxidation and dissected its necessity. Liquid chromatography-mass spectrometry (LC-MS/MS) analysis identified three lysine residues (K116, K2391, K2401) that were specifically oxidized by both LOXL2 and LOXL3 (FIG. 4A). Since no active recombinant LOX enzyme is available, HASMC were cultured and the FN they secrete was subjected to LC-MS/MS analysis. In a similar manner to the two LOX-like proteins, LOX was also shown to specifically oxidize lysine 116 (FIG. 4A and FIG. 7A), altogether demonstrating the specificity of the reaction and reinforcing the above observations of the shared FN-dependent activities the LOX enzymes carry. Analysis of the oxidized lysine residues demonstrates that they are all localized to FN type 1 (FN1) repeats (FN1 repeat #2 and FN repeat #12), one close to the FN N-terminus and the other in its C-terminal region. Both regions encompassing these lysine residues are highly conserved in vertebrate FN (FIG. 4B) suggesting they play an important role in FN activity.

To directly test the role that these three lysine residues play in cell adhesion and FN fibrillogenesis, a lentiviral construct was generated expressing a mutant FN form where K116, K2391, K2401 were mutated to Alanines, rendering them incompetent for oxidation (FN^{ΔOxi}). As a control, the present inventors mutated the α5β1 and αv integrins binding site RGD to RGE (FN^{ΔRGD}) thus blocking their binding through this site (39). Western blot analysis from cells overexpressing these two constructs demonstrated that they are expressed to similar levels as that of cells over-expressing the full length wild-type FN protein (FN^{WT}) (FIG. 4C-D).

Considering that LOX treatment enhances FN clustering (FIGS. 2A-M), the present inventors took into account that under classical adhesion assays with excess FN adsorbed to the surface (typically 10-20 μg/ml) stochastic clustering readily occurs. Thus, to avoid the possible inference of pre-adsorbed FN, early adhesion assays of the cells expressing the FN variants were performed directly on glass coverslips with no FN coating. Cells were cultured for 1 hour and their areas were quantified to test whether the non-oxidizable FN variant inhibits initiation of cell adhesion. It was found that the over-expression of FN^{ΔRGD} led to a significant reduction in cell area and adhesion sizes, demonstrating the inhibition of the FN-integrin interactions. Notably, the over-expression of FN^{ΔOxi} led to a similar reduction in cell area and adhesion sizes as that observed with the over-expression of FN^{ΔRGD}, demonstrating the role these oxidation sites play in the initiation of FN-dependent integrin activation (FIG. 4E-I).

To explore whether these mutations affect also FN fibrillogenesis, the present inventors first tested whether cells fibrilize their own secreted FN. To that end, coverslips were coated with Alexa 647-labelled FN and MEF FN-GFP (in which GFP is knocked-in to both alleles of the FN gene (24)) and cultured for 48 hours, followed by imaging of the FN fibrils. All FN fibrils that were observed were GFP labelled, demonstrating that cells use their own secreted FN in forming the initial fibrils (FIG. 7B-C). Next, the cells expressing the FN variants were cultured for 24 hours, immunostained for FN, and FN fibrillation was monitored. Cells over-expressing the FN^{ΔRGD} demonstrated a significant 27% reduction in the ratio between fibrillar FN vs. total FN; thus, even though integrin binding was perturbed in the lack of the RGD site, alternative interactions with αvβ3 integrin receptors could promote FN fibrillogenesis (39), but to a lesser extent. Strikingly, cells over-expressing FN^{ΔOxi} displayed significantly less fibrillar FN (>62% reduction), despite containing the wild-type RGD site, demonstrating that the oxidation is a prerequisite for adhesion initiation (FIG. 4J-M). As these cells still express endogenous wild-type FN, these results strongly demonstrate the critical and essential role played by members of the LOX-family enzymes in regulating FN fibrillogenesis, establishing that initiation of the process is an enzyme—rather than force-dependent process. This is in line with the observation that even when FN^{ΔOxi}-expressing cells generate force-bearing stress fibers, no FN fibrils appear beneath them (FIG. 4N). Finally, it was found that whereas HASMC infected with shCtrl lentiviral particles nicely form FN fibrils when cultured directly on glass, shLOX HASMC do not form any FN fibrils, even though they were cultured on a very stiff matrix and even though they too formed stress fibers (FIG. 4O-R), demonstrating the necessity for LOX in regulating FN fibrillogenesis.

The present results reveal a novel regulatory step essential for the formation of FN fibrils. The enzymatic activity of lysyl oxidases leads to oxidation of specific lysine residues within FN, resulting in a structural change in the FN dimers thereby promoting early adhesion. It may be proposde that through this structural change, a local increase in RGD density occurs, thus serving as a nucleator of FN fibrillogenesis. Following this nucleation step, integrin-mediated cell adhesion is favored, leading to increased force transmission to the matrix and the subsequent formation of bona-fide FN fibrils (FIG. 4S). This process is particularly important for cells to overcome the soft embryonic environment through local secretion of LOX enzymes along migration routes, e.g. in the case of neural crest cells. Thus, similar to nucleation of cytoskeletal fibers' polymerization (e.g. F-actin), which is regulated locally through the activity of nucleators (e.g. formins), initiation of FN fibrillogenesis also requires nucleation. As this nucleation step is an enzyme-dependent process, rather than a force-dependent one, these observations bridge the observed discrepancies between the in vivo observations of FN fibrils in early embryos and in soft tissues, and the cell culture-based models which suggest that fibrillogenesis occurs primarily on stiff matrices.

The present results suggest that in the lack of LOX activity, cells perceive a soft environment even on rigid matrices. The association between LOX activity and rigidity perception highlight an alternative explanation to the observed association of lysyl oxidase upregulation and metastases (40). Thus, cells initiate migration not only in response to general tissue stiffness but rather due to their local immediate environment, or the perception of their environment, even on soft matrices. As the majority of adhesion assays are carried out on FN coated matrices, it may be suggested that such conditions mask the LOX-dependent nucleation step and alter the perception of the immediate outside environment. The present results therefore imply that to identify and study the early steps of cell adhesion, cells must be cultured on matrices with either no FN or limiting FN levels. The ubiquitous expression of FN, and the ability of the various widely expressed members of the LOX family to oxidize it, suggest that the process that has been identified is general and is not specific to a distinct tissue or cell type. This process is thus expected to have important implications for the present understanding of various matrix-dependent cellular functions in different tissues and contexts.

### Inhibitor Peptides Block Fibronectin Fibrillogenesis

The following inhibitor peptides were used in the experiments:

FN1-2 (Inhibitor peptide):
(SEQ ID NO: 19)
ETCFDKYTGNTYRVGDTYERPKDSMIWDCTCIGAGRGRISCTIA
FN1-12 (Inhibitor peptide):
(SEQ ID NO: 20)
ATCYDDGKTYHVGEQWQKEYLGAICSCTCFGGQRGWRCDNCRRP
FN1-3 (control peptide):
(SEQ ID NO: 21)
NRCHEGGQSYKIGDTWRRPHETGGYMLECVCLGNGKGEWTCKPI
FN1-9 (control peptide):
(SEQ ID NO: 22)
DQCQDSETGTFYQIGDSWEKYVHGVRYQCYCYGRGIGEWHCQPL
FN1-2 mut (Inhibitor peptide):
(SEQ ID NO: 23)
ETCFDKYTGNTYRVGDTYERPRDSMIWDCTCIGAGRGRISCTIA
FN1-12 mut (Inhibitor peptide):
(SEQ ID NO: 24)
ATCYDDGRTYHVGEQWQREYLGAICSCTCFGGQRGWRCDNCRRP.

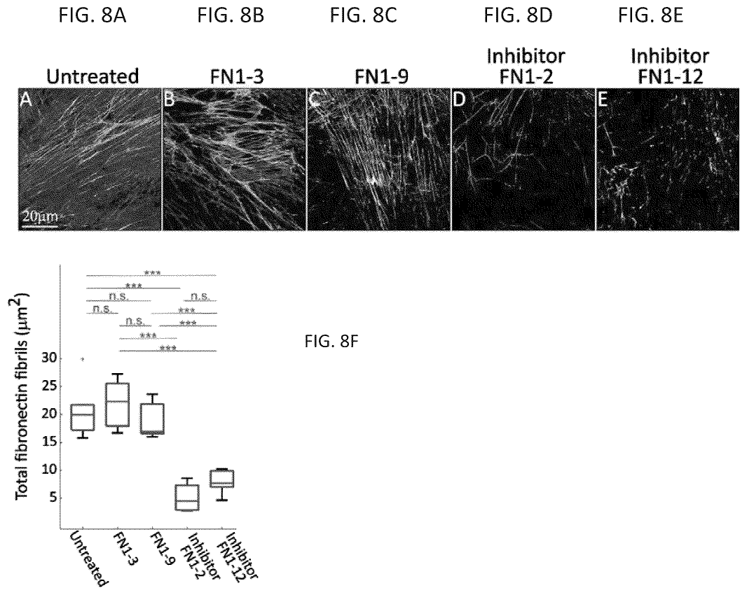

Representative images (FIGS. 8A-E) of fibronectin immunostaining (color-coded for intensity) underneath human dermal fibroblast (HDF) cells exposed to the different peptides (8B—SEQ ID NO: 21, 8C—SEQ ID NO: 22; 8D—SEQ ID NO: 19; 8E—SEQ ID NO: 20), seeded on glass without external FN coating and fixed after 1 week (A-E, scale=20 μm) illustrate that inhibitor peptides block fibronectin fibrillogenesis. A quantitative summary of the total fibronectin fibrils is shown in FIG. 8F which demonstrates the reduction in fibrils with exposure to the different inhibitors (FN1-2 and FN1-12), as compared with the control peptides (FN1-3 and FN1-9) or the untreated sample.

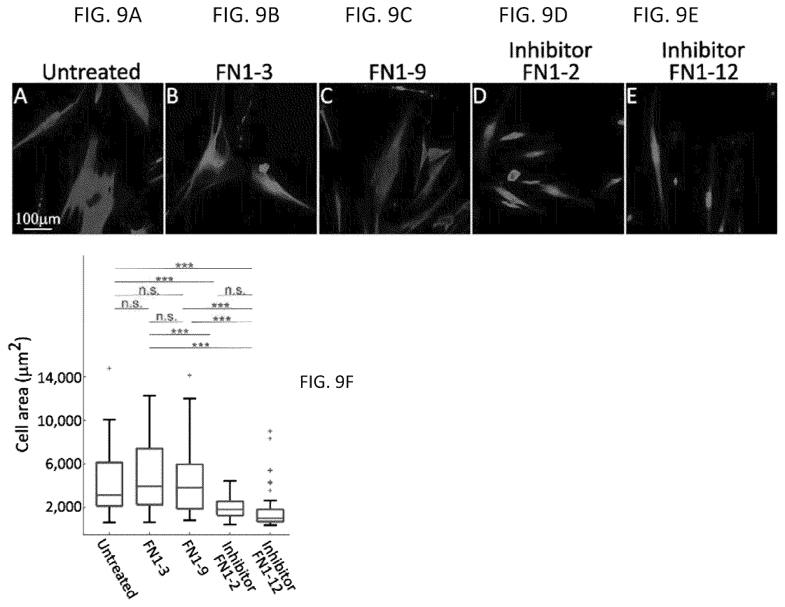

Representative images (FIGS. 9A-E) of HDF cell contour following exposure to the different peptides (9B—SEQ ID NO: 21, 9C—SEQ ID NO: 22; 9D—SEQ ID NO: 19; 9E—SEQ ID NO: 20), seeded on glass without external FN coating and fixed after 1 week (A-E, scale=100 μm) illustrate that inhibitor peptides reduce cell size. A quantitative summary of the cell area, showing significant reduction with exposure to the different inhibitor peptides (FN1-2 and FN-1-12), as compared with the control peptides (FN1-3 and FN1-9) or the untreated sample is illustrated in FIG. 9F.

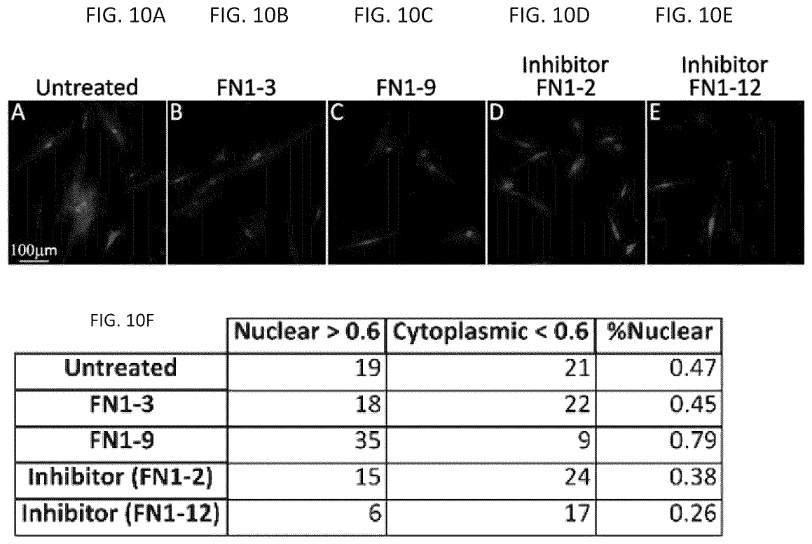

Representative images (FIGS. 10A-E) of immunofluorescence for Yap, as a readout of mechanosensation in HDF cells following culturing with the different peptides (10B—SEQ ID NO: 21, 10C—SEQ ID NO: 22; 10D—SEQ ID NO: 19; 10E—SEQ ID NO: 20), seeded on glass without external FN coating and fixed after 1 week (A-E, scale=100 μm) illustrate that inhibitor peptides affect cellular mechanosensation.

FIG. 10F provides a table which summarizes the number of cells with nuclear or cytoplasmic Yap, as well as the percent of the nuclear protein.

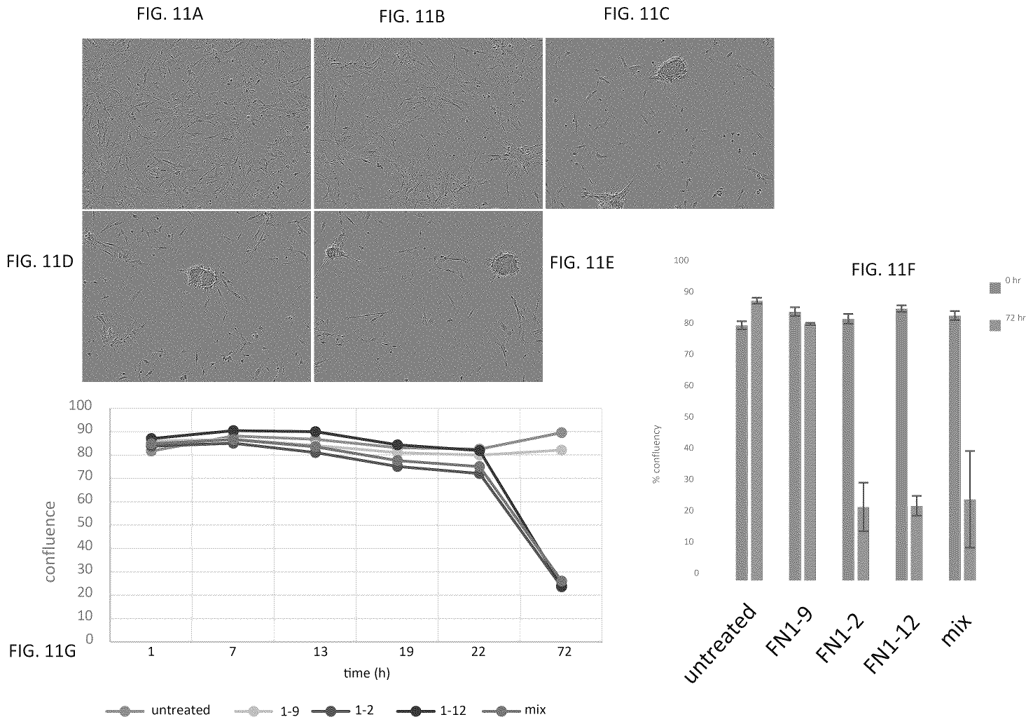

Representative images of lung fibroblasts following 72 hours in culture without peptide addition (A), control peptide (FN1-9; B) or inhibitor peptides (FN1-2, FN1-12 or both of them; C-E, respectively) are shown in FIGS. 11A-E. Quantification of culture confluency following 72 hours demonstrates the inhibitor peptides (FN1-2 and FN1-12) or both (mix) but not with the control peptide (FN1-9) inhibit cell proliferation (F). Note the lack of effect in the first day of culturing (G).

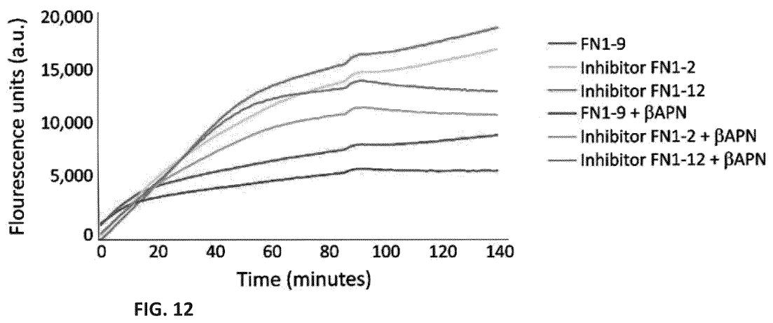

As illustrated in FIG. 12, inhibitor peptides undergo oxidation by Lysyl oxidases. Oxidation assay demonstrates the inhibitor peptides (FN1-2 and FN1-12) but not the control peptides (FN1-3 and FN1-9) undergo oxidation by LOXL3. This oxidation is inhibited following the addition of βAPN (LOX family competitive inhibitor).

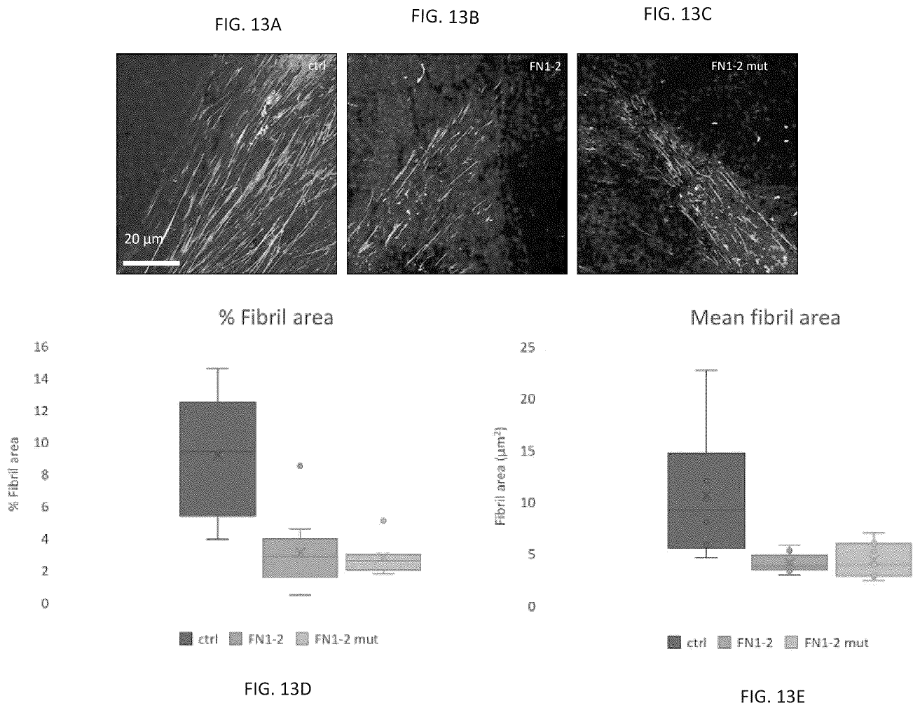

Inhibitor Fc-fused peptides (SEQ ID NOs: 28, 29) were shown to affect fibronectin fibrillogenesis. Representative images of fibronectin immunostaining (color-coded for intensity) underneath human dermal fibroblasts (HDF) seeded on glass without external FN coating and fixed after 1 week are shown in FIGS. 13A-C. The cells were either left without Fc-fused peptide addition (FIG. 13A), with Fc-fused inhibitor peptide FN1-2 (FIG. 13B), or with Fc-fused inhibitor peptide FN1-2 mutated at the oxidation site 116 (lys-to-arg) (SEQ ID NO: 30; FIG. 13C). Boxplot representing the ratio between the total area of fibronectin fibrils longer than 5 μm and the total measured area in each image is shown in FIG. 13D. This boxplot shows the significant reduction in the percentage of long fibrils when cells are exposed to the FC-fused inhibitor peptides. Boxplots of the mean area of fibrils in each condition, demonstrating the significant decrease in fibrils length following exposure to the FC-fused inhibitor peptides are shown in FIG. 13E.

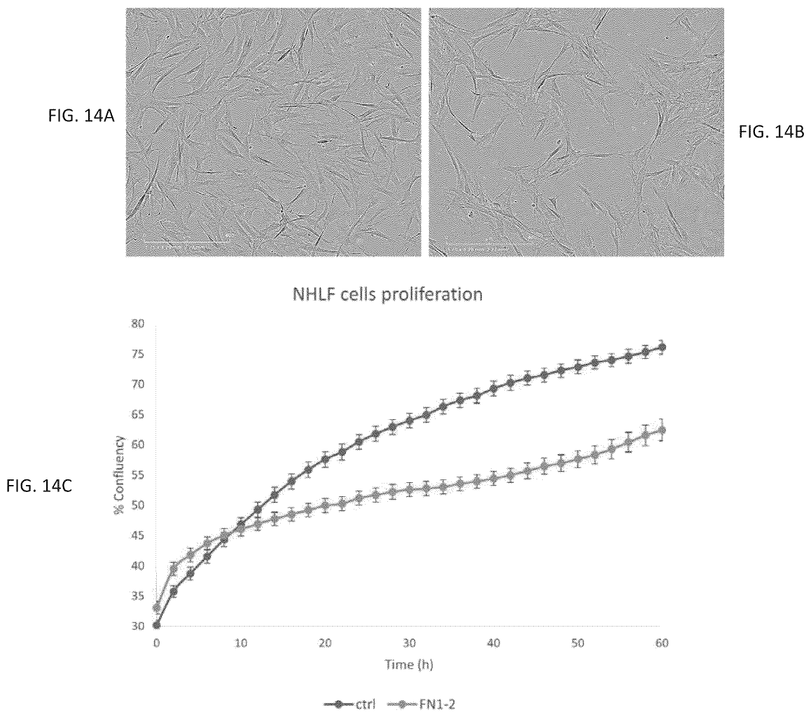

Representative images of normal human lung fibroblasts (NHLF) following 48 hours in culture, without Fc-fused peptide addition (FIG. 14A) or with Fc-fused inhibitor peptide FN1-2 (FIG. 14B). Quantification of culture confluency over time demonstrates the inhibitor Fc-fused peptide FN1-2 inhibits cell proliferation is provided in FIG. 14C.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

ADDIN Mendeley Bibliography CSL_BIBLIOGRAPHY 1. C. Bonnans, J. Chou, Z. Werb, Remodelling the extracellular matrix in development and disease. Nat. Rev. Mol. Cell Biol. 15, 786-801 (2014).
2. J. D. Humphrey, E. R. Dufresne, M. A. Schwartz, Mechanotransduction and extracellular matrix homeostasis. Nat. Rev. Mol. Cell Biol. 15, 802-812 (2014).
3. J. E. Schwarzbauer, D. W. DeSimone, Fibronectins, their fibrillogenesis, and in vivo functions. Cold Spring Harb. Perspect. Biol. 3, 1-19 (2011).
4. R. Pankov, K. M. Yamada, Fibronectin at a glance. J. Cell Sci. 115, 3861-3863 (2002).
5. L. I. Gold, E. Pearlstein, Fibronectin-collagen binding and requirement during cellular adhesion. Biochem. J. 186, 551-559 (1980).
6. L. Sabatier, D. Chen, C. Fagotto-Kaufmann, D. Hubmacher, M. D. McKee, D. S. Annis, D. F. Osher, D. P. Reinhardt, Fibrillin assembly requires fibronectin. Mol. Biol. Cell. 20, 846-858 (2009).
7. M. Pereira, B. J. Rybarczyk, T. M. Odrljin, D. C. Hocking, J. Sottile, P. J. Simpson-Haidaris, The incorporation of fibrinogen into extracellular matrix is dependent on active assembly of a fibronectin matrix. J. Cell Sci. 115, 609-617 (2002).
8. C. Y. Chung, H. P. Erickson, Glycosaminoglycans modulate fibronectin matrix assembly and are essential for matrix incorporation of tenascin-C. J. Cell Sci. 110, 1413-1419 (1997).
9. P. Singh, C. Carraher, J. E. Schwarzbauer, Assembly of Fibronectin Extracellular Matrix. http://dx(dot)doi(dot)org/10.1146/annurev-cellbio-100109-104020. 26, 397-419 (2010).
10. S. M. Friih, I. Schoen, J. Ries, V. Vogel, Molecular architecture of native fibronectin fibrils. Nat. Commun. 6 (2015), doi:10.1038/ncomms8275.
11. C. Zhong, M. Chrzanowska-Wodnicka, J. Brown, A. Shaub, A. M. Belkin, K. Burridge, Rho-mediated contractility exposes a cryptic site in fibronectin and induces fibronectin matrix assembly. J. Cell Biol. 141, 539-551 (1998).
12. M. Antia, G. Baneyx, K. E. Kubow, V. Vogel, Fibronectin in aging extracellular matrix fibrils is progressively unfolded by cells and elicits an enhanced rigidity response. Faraday Discuss. 139, 229-249 (2008).
13. L. Cao, J. Nicosia, J. Larouche, Y. Zhang, H. Bachman, A. C. Brown, L. Holmgren, T. H. Barker, Detection of an Integrin-Binding Mechanoswitch within Fibronectin during Tissue Formation and Fibrosis. ACS Nano. 11, 7110 (2017).
14. C. L. Carraher, J. E. Schwarzbauer, Regulation of matrix assembly through rigidity-dependent fibronectin conformational changes. J. Biol. Chem. 288, 14805-14814 (2013).
15. G. Baneyx, L. Baugh, V. Vogel, Fibronectin extension and unfolding within cell matrix fibrils controlled by cytoskeletal tension. Proc. Natl. Acad. Sci. U.S.A 99, 5139-5143 (2002).
16. E. Zamir, M. Katz, Y. Posen, N. Erez, K. M. Yamada, B.-Z. Katz, S. Lin, D. C. Lin, A. Bershadsky, Z. Kam, B. Geiger, Dynamics and segregation of cell-matrix adhesions in cultured fibroblasts. Nat. Cell Biol. 2 (2000).
17. H. Wolfenson, I. Lavelin, B. Geiger, Dynamic Regulation of the Structure and Functions of Integrin Adhesions. Dev. Cell. 24, 447-458 (2013).
18. B. J. Dzamba, K. R. Jakab, M. Marsden, M. A. Schwartz, D. W. DeSimone, Cadherin Adhesion, Tissue Tension, and Noncanonical Wnt Signaling Regulate Fibronectin Matrix Organization. Dev. Cell. 16, 421-432 (2009).
19. U. Agero, J. A. Glazier, M. Hosek, Bulk elastic properties of chicken embryos during somitogenesis. Biomed. Eng. Online. 9, 1-16 (2010).
20. M. Marrese, N. Antonovaite, B. K. A. Nelemans, T. H. Smit, D. Iannuzzi, Micro-indentation and optical coherence tomography for the mechanical characterization of embryos: Experimental setup and measurements on chicken embryos. Acta Biomater. 97, 524-534 (2019).
21. P. Rifes, S. Thorsteinsdóttir, Extracellular matrix assembly and 3D organization during paraxial mesoderm development in the chick embryo. Dev. Biol. 368, 370-381 (2012).
22. P. G. de Almeida, G. G. Pinheiro, A. M. Nunes, A. B. Gongalves, S. Thorsteinsdóttir, Fibronectin assembly during early embryo development: A versatile communication system between cells and tissues. Dev. Dyn. 245, 520-535 (2016).
23. O. Kraft-Sheleg, S. Zaffryar-Eilot, O. Genin, W. Yaseen, S. Soueid-Baumgarten, O. Kessler, T. Smolkin, G. Akiri, G. Neufeld, Y. Cinnamon, P. Hasson, Localized LoxL3-Dependent Fibronectin Oxidation Regulates Myofiber Stretch and Integrin-Mediated Adhesion. Dev. Cell. 36, 550-561 (2016).
24. D. Tomer, C. Arriagada, S. Munshi, B. E. Alexander, B. French, P. Vedula, V. Caorsi, A. House, M. Guvendiren, A. Kashina, J. E. Schwarzbauer, S. Astrof, A new mechanism of fibronectin fibril assembly revealed by live imaging and super-resolution microscopy. bioRxiv, https://doi(dot)org/10.1101/2020.09.09.290130.
25. X. Wang, S. Astrof, Neural crest cell-autonomous roles of fibronectin in cardiovascular development. Dev. 143, 88-100 (2016).
26. A. Mittal, M. Pulina, S. Y. Hou, S. Astrof, Fibronectin and integrin alpha 5 play essential roles in the development of the cardiac neural crest. Mech. Dev. 127, 472-484 (2010).
27. T. Matsumoto, S. Sugita, K. Nagayama, K. Tanishita, K. Yamamoto, Eds. (Springer Japan, Tokyo, 2016; https://doi(dot)org/10.1007/978-4-431-54801—O_7), pp. 127-140.
28. L. V. S., H. C. M., H. E. P., C. Nikkola, L. Ignaty, L. C. G., B. A. J., V. Dana, null null, M. R. P., F. N. Y., S. N. O., M. Richard, S. Shamil, V. Dana, K. Joel, A. Aaron, B. Corneliu, B. Andrew, C. Nikkola, C. Christopher, F. N. Y., G. Robert, G. Wolfram, H. Alireza, H. Jason, K. Elizabeth, M. Calum, M. Barbara, M. Anwoy, O. Melanie, R. Soumya, S. Christine, S. Sheila, V. Jamie, Q. Haiyan, Loss of function mutation in LOX causes thoracic aortic aneurysm and dissection in humans. Proc. Natl. Acad. Sci. 113, 8759-8764 (2016).
29. A. Sampath Narayanan, R. C. Siegel, G. R. Martin, On the inhibition of lysyl oxidase by p3-aminopropionitrile. Biochem. Biophys. Res. Commun. 46, 745-751 (1972).
30. H. Wolfenson, G. Meacci, S. Liu, M. R. M. R. Stachowiak, T. Iskratsch, S. Ghassemi, P. Roca-Cusachs, B. O'Shaughnessy, J. Hone, M. P. M. P. Sheetz, Tropomyosin controls sarcomere-like contractions for rigidity sensing and suppressing growth on soft matrices. Nat. Cell Biol. 18, 33-42 (2016).
31. L. Feld, L. Kellerman, A. Mukherjee, A. Livne, E. Bouchbinder, H. Wolfenson, Cellular contractile forces are nonmechanosensitive. Sci. Adv. 6 (2020), doi:10.1126/SCIADV.AAZ6997.
32. S. Ghassemi, G. Meacci, S. Liu, A. A. Gondarenko, A. Mathur, P. Roca-Cusachs, M. P. Sheetz, J. Hone, Cells test substrate rigidity by local contractions on submicrometer pillars. Proc. Natl. Acad. Sci. U.S.A 109, 5328-5333 (2012).
33. M. Ghibaudo, A. Saez, L. Trichet, A. Xayaphoummine, J. Browaeys, P. Silberzan, A. Buguin, B. Ladoux, Traction forces and rigidity sensing regulate cell functions. Soft Matter. 4, 1836 (2008).
34. G. Jiang, G. Giannone, D. R. Critchley, E. Fukumoto, M. P. Sheetz, Two-piconewton slip bond between fibronectin and the cytoskeleton depends on talin. Nature. 424, 334-337 (2003).
35. M. Schvartzman, M. Palma, J. Sable, J. Abramson, X. Hu, M. P. Sheetz, S. J. Wind, Nanolithographic control of the spatial organization of cellular adhesion receptors at the single-molecule level. Nano Lett. 11, 1306-1312 (2011).
36. E. A. Cavalcanti-Adam, T. Volberg, A. Micoulet, H. Kessler, B. Geiger, J. P. Spatz, Cell spreading and focal adhesion dynamics are regulated by spacing of integrin ligands. Biophys. J. 92, 2964-2974 (2007).
37. S. D. Vallet, C. Berthollier, R. Salza, L. Muller, S. Ricard-Blum, The Interactome of Cancer-Related Lysyl Oxidase and Lysyl Oxidase-Like Proteins. Cancers 2021, Vol. 13, Page 71. 13, 71 (2020).
38. J. T. Parsons, A. R. Horwitz, M. A. Schwartz, Cell adhesion: integrating cytoskeletal dynamics and cellular tension. Nat. Rev. Mol. Cell Biol. 2010 119. 11, 633-643 (2010).
39. S. Takahashi, M. Leiss, M. Moser, T. Ohashi, T. Kitao, D. Heckmann, A. Pfeifer, H. Kessler, J. Takagi, H. P. Erickson, R. Fassler, The RGD motif in fibronectin is essential for development but dispensable for fibril assembly. J. Cell Biol. 178, 167-178 (2007).
40. P. G. Amendola, R. Reuten, J. T. Erler, Interplay Between LOX Enzymes and Integrins in the Tumor Microenvironment. Cancers (Basel). 11 (2019), doi:10.3390/CANCERS11050729.
41. I. Arganda-Carreras, V. Kaynig, C. Rueden, K. W. Eliceiri, J. Schindelin, A. Cardona, H. Sebastian Seung, Trainable Weka Segmentation: a machine learning tool for microscopy pixel classification. Bioinformatics. 33, 2424-2426 (2017).
42. E. Gutierrez, A. Groisman, Measurements of elastic moduli of silicone gel substrates with a microfluidic device. PLoS One. 6 (2011), doi:10.1371/journal.pone.0025534.
43. L. Nahidiazar, A. V. Agronskaia, J. Broertjes, B. Den Van Broek, K. Jalink, Optimizing imaging conditions for demanding multi-color super resolution localization microscopy. PLoS One. 11, 1-18 (2016).
44. A. Edelstein, N. Amodaj, K. Hoover, R. Vale, N. Stuurman, Curr Protoc Mol Biol 92, in press, doi:10.1002/0471142727.mbl420s92.
45. R. Kalev-Altman, E. Hanael, E. Zelinger, M. Blum, E. Monsonego-Ornan, D. Sela-Donenfeld, Conserved role of matrix metalloproteases 2 and 9 in promoting the migration of neural crest cells in avian and mammalian embryos. FASEB J. 34, 5240-5261 (2020).

What is claimed is:

1. A method of treating a disease associated with fibrosis in a subject in need thereof comprising administering to the subject a therapeutically effective amount of an isolated peptide comprising an amino acid sequence of a lysyl oxidase (LOX) family member target site of an extracellular matrix protein, said peptide being no longer than 50 amino acids, thereby treating the disease associated with fibrosis.

2. The method according to claim 1, wherein said peptide comprises a modification which imparts said peptide with enhanced stability under physiological conditions as compared to a native form of said peptide not comprising said modification.

3. The method according to claim 2, wherein said modification comprises a proteinaceous modification.

4. The method according to claim 3, wherein said proteinaceous modification is selected from the group consisting of immunoglobulin, human serum albumin, and transferrin.

5. The method according to claim 4, wherein said immunoglobulin comprises an Fc domain.

6. The method according to claim 1, wherein said LOX family member is selected from the group consisting of LOX, LOXL1, LOXL2, LOXL3 and LOXL4.

7. The method according to claim 1, wherein said extracellular matrix protein is encoded by a gene selected from the group consisting of ADAMTS10, ADAMTS14, ADAMTS2, ADAMTSL2, ADAMTSL4, ANGPTL4, COL17A1, COL18A1, COL1A1, COL1A2, COL3A1, COL4A1, COL4A2, COL5A1, COL6A1, COL6A3, CTGF, ECM1, EGFL7, ELN, EPHA7, FBLN5, FBN1, FGF2, FMOD, FN1, ITGA5, ITGB1, LAMB1, LAMB2, LAMC1, LTBP1, MFAP4, MFAP5, NID1, PDGFRA, SPARC, THBS1, TIMP3 and TNC.

8. The method according to claim 1, wherein said extracellular matrix protein is fibronectin.

9. The method according to claim 8, wherein said peptide comprises at least one of the sequences selected from the group consisting of SEQ ID NO: 12 (RPKDS), SEQ ID NO: 13 (DGKTY), SEQ ID NO: 14 (WQKEY) and SEQ ID NO: 15 (ERPKDSM).

10. The method according to claim 1, wherein said peptide comprises an amino acid sequence at least 90% identical to SEQ ID NO: 17 or SEQ ID NO: 18.

11. The method according to claim 1, wherein said disease is cancer and Duchenne muscular dystrophy (DMD).

12. The method according to claim 1, wherein said disease is associated with liver fibrosis, cardiac fibrosis or lung fibrosis.

13. An isolated peptide comprising an amino acid sequence of a lysyl oxidase (LOX) family member target site of an extracellular matrix protein, said peptide being no longer than 50 amino acids, wherein said peptide comprises a modification which imparts said peptide with enhanced stability under physiological conditions as compared to a native form of said peptide not comprising said modification.

14. The isolated peptide of claim 13, wherein said extracellular matrix protein is encoded by a gene selected from the group consisting of ADAMTS10, ADAMTS14, ADAMTS2, ADAMTSL2, ADAMTSL4, ANGPTL4, COL17A1, COL18A1, COL1A1, COL1A2, COL3A1, COL4A1, COL4A2, COL5A1, COL6A1, COL6A3, CTGF, ECM1, EGFL7, ELN, EPHA7, FBLN5, FBN1, FGF2, FMOD, FN1, ITGA5, ITGB1, LAMB1, LAMB2, LAMC1, LTBP1, MFAP4, MFAP5, NID1, PDGFRA, SPARC, THBS1, TIMP3 and TNC.

15. The isolated peptide of claim 13, wherein said extracellular matrix protein is fibronectin.

16. The isolated peptide of claim 13, wherein said modification comprises a proteinaceous modification.

17. The isolated peptide of claim 15, wherein said peptide comprises at least one of the sequences selected from the group consisting of SEQ ID NO: 12 (RPKDS), SEQ ID NO: 13 (DGKTY), SEQ ID NO: 14 (WQKEY) and SEQ ID NO: 15 (ERPKDSM).

18. The isolated peptide of claim 13, being no longer than 20 amino acids, in the absence of said modification.

19. The isolated peptide of claim 13, being no longer than 400 amino acids.

20. A pharmaceutical composition comprising the peptide of claim 13 as the active agent, and a pharmaceutically acceptable carrier, being formulated for local delivery or systemic delivery.


SEQUENCE LISTING (46 sequences)

SEQ ID NO: 1 - DNA, 20 bp, synthetic construct
  misc_feature 1..20: note = Single strand DNA oligonucleotide
  source 1..20: mol_type = other DNA
gccagggtgaagtgaaagac

SEQ ID NO: 2 - DNA, 20 bp, synthetic construct
  misc_feature 1..20: note = Single strand DNA oligonucleotide
  source 1..20: mol_type = other DNA
gatctgggatgctgaagacc

SEQ ID NO: 3 - DNA, 22 bp, synthetic construct
  misc_feature 1..22: note = Single strand DNA oligonucleotide
  source 1..22: mol_type = other DNA
gaacttcggaataggaacttcg

SEQ ID NO: 4 - DNA, 22 bp, synthetic construct
  misc_feature 1..22: note = Single strand DNA oligonucleotide
  source 1..22: mol_type = other DNA
tgagcgtcctgcagactccatg

SEQ ID NO: 5 - DNA, 18 bp, synthetic construct
  misc_feature 1..18: note = Single strand DNA oligonucleotide
  source 1..18: mol_type = other DNA
taagtgtcacccactcgg

SEQ ID NO: 6 - DNA, 21 bp, synthetic construct
  misc_feature 1..21: note = Single strand DNA oligonucleotide
  source 1..21: mol_type = other DNA
gccgtggagaaagccccgcaa

SEQ ID NO: 7 - DNA, 34 bp, synthetic construct
  misc_feature 1..34: note = Single strand DNA oligonucleotide
  source 1..34: mol_type = other DNA
cagtgacagcatacacagtgatggtataatcaac

SEQ ID NO: 8 - DNA, 23 bp, synthetic construct
  misc_feature 1..23: note = Single strand DNA oligonucleotide
  source 1..23: mol_type = other DNA
tgatgatggggcgacataccacg

SEQ ID NO: 9 - DNA, 19 bp, synthetic construct
  misc_feature 1..19: note = Single strand DNA oligonucleotide
  source 1..19: mol_type = other DNA
taacacgttgcctcatgag

SEQ ID NO: 10 - DNA, 23 bp, synthetic construct
  misc_feature 1..23: note = Single strand DNA oligonucleotide
  source 1..23: mol_type = other DNA
acagtggcaggcggaatatctcg

SEQ ID NO: 11 - DNA, 18 bp, synthetic construct
  misc_feature 1..18: note = Single strand DNA oligonucleotide
  source 1..18: mol_type = other DNA
tctcctacgtggtatgtc

SEQ ID NO: 12 - protein, 5 aa, synthetic construct
  REGION 1..5: note = peptide sequence
RPKDS

SEQ ID NO: 13 - protein, 5 aa, synthetic construct
  REGION 1..5: note = peptide sequence
DGKTY

SEQ ID NO: 14 - protein, 5 aa, synthetic construct
  REGION 1..5: note = peptide sequence
WQKEY

SEQ ID NO: 15 - protein, 7 aa, synthetic construct
  REGION 1..7: note = peptide sequence
ERPKDSM

SEQ ID NO: 16 - protein, 2477 aa, Homo sapiens
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 17 - protein, 31 aa, synthetic construct
  REGION 1..31: note = short version of FN1-2
YTGNTYRVGDTYERPKDSMIWDCTCIGAGRG

SEQ ID NO: 18 - protein, 31 aa, synthetic construct
  REGION 1..31: note = short version of FN1-12
CYDDGKTYHVGEQWQKEYLGAICSCTCFGGQ

SEQ ID NO: 19 - protein, 44 aa, synthetic construct
  REGION 1..44: note = FN1-2 (Inhibitor peptide) long version of FN1-2
ETCFDKYTGNTYRVGDTYERPKDSMIWDCTCIGAGRGRISCTIA

SEQ ID NO: 20 - protein, 44 aa, synthetic construct
  REGION 1..44: note = FN1-12 (Inhibitor peptide long version)
ATCYDDGKTYHVGEQWQKEYLGAICSCTCFGGQRGWRCDNCRRP

SEQ ID NO: 21 - protein, 44 aa, synthetic construct
  REGION 1..44: note = FN1-3 (control peptide)
NRCHEGGQSYKIGDTWRRPHETGGYMLECVCLGNGKGEWTCKPI

SEQ ID NO: 22 - protein, 44 aa, synthetic construct
  REGION 1..44: note = FN1-9 (control peptide)
DQCQDSETGTFYQIGDSWEKYVHGVRYQCYCYGRGIGEWHCQPL

SEQ ID NO: 23 - protein, 44 aa, synthetic construct
  REGION 1..44: note = FN1-2 mut (Inhibitor peptide)
ETCFDKYTGNTYRVGDTYERPRDSMIWDCTCIGAGRGRISCTIA

SEQ ID NO: 24 - protein, 44 aa, synthetic construct
  REGION 1..44: note = FN1-12 mut (Inhibitor peptide)
ATCYDDGRTYHVGEQWQREYLGAICSCTCFGGQRGWRCDNCRRP

SEQ ID NO: 25 - protein, 50 aa, synthetic construct
  REGION 1..50: note = Free peptides
KKKETCFDKYTGNTYRVGDTYERPKDSMIWDCTCIGAGRGRISCTIAKKK

SEQ ID NO: 26 - protein, 50 aa, synthetic construct
  REGION 1..50: note = Free peptides
KKKATCYDDGKTYHVGEQWQKEYLGAICSCTCFGGQRGWRCDNCRRPKKK

SEQ ID NO: 27 - protein, 232 aa, synthetic construct
  REGION 1..232: note = Fc region
DPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 276 aa, synthetic construct
  REGION 1..276: note = FN1-2 domain connected to Fc
ETCFDKYTGNTYRVGDTYERPKDSMIWDCTCIGAGRGRISCTIADPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 277 aa, synthetic construct
  REGION 1..277: note = FN1-12 domain connected to Fc (with a short linker)
ATCYDDGKTYHVGEQWQKEYLGAICSCTCFGGQRGWRCDNCRRPADPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 276 aa, synthetic construct
  REGION 1..276: note = FN1-12 domain connected to Fc (with a short linker)
ETCFDKYTGNTYRVGDTYERPRDSMIWDCTCIGAGRGRISCTIADPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 277 aa, synthetic construct
  REGION 1..277: note = mutated form of FN1-12 domain connected to Fc (with a short linker)
ATCYDDGRTYHVGEQWQREYLGAICSCTCFGGQRGWRCDNCRRPADPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 41 aa, synthetic construct
  REGION 1..41: note = XXXXX
PGCYDNGKHYQINQQWERTYLGNALVCTCYGGSRGFNCESK

SEQ ID NO: 33 - protein, 44 aa, synthetic construct; identical to SEQ ID NO: 19
  REGION 1..44: note = FN1 domains from fibronectin

SEQ ID NO: 34 - protein, 44 aa, synthetic construct; identical to SEQ ID NO: 21
  REGION 1..44: note = FN1 domains from fibronectin

SEQ ID NO: 35 - protein, 45 aa, synthetic construct
  REGION 1..45: note = FN1 domains from fibronectin
EKCFDHAAGTSYVVGETWEKPYQGWMMVDCTCLGEGSGRITCTSR

SEQ ID NO: 36 - protein, 45 aa, synthetic construct
  REGION 1..45: note = FN1 domains from fibronectin
NRCNDQDTRTSYRIGDTWSKKDNRGNLLQCICTGNGRGEWKCERH

SEQ ID NO: 37 - protein, 40 aa, synthetic construct
  REGION 1..40: note = FN1 domains from fibronectin
GHCVTDSGVVYSVGMQWLKTQGNKQMLCTCLGNGVSCQET

SEQ ID NO: 38 - protein, 44 aa, synthetic construct
  REGION 1..44: note = FN1 domains from fibronectin
EICTTNEGVMYRIGDQWDKQHDMGHMMRCTCVGNGRGEWTCIAY

SEQ ID NO: 39 - protein, 43 aa, synthetic construct
  REGION 1..43: note = FN1 domains from fibronectin
DQCIVDDITYNVNDTFHKRHEEGHMLNCTCFGQGRGRWKCDPV

SEQ ID NO: 40 - protein, 44 aa, synthetic construct; identical to SEQ ID NO: 22
  REGION 1..44: note = FN1 domains from fibronectin

SEQ ID NO: 41 - protein, 45 aa, synthetic construct
  REGION 1..45: note = FN1 domains from fibronectin
DSCFDPYTVSHYAVGDEWERMSESGFKLLCQCLGFGSGHFRCDSS

SEQ ID NO: 42 - protein, 43 aa, synthetic construct
  REGION 1..43: note = FN1 domains from fibronectin
RWCHDNGVNYKIGEKWDRQGENGQMMSCTCLGNGKGEFKCDPH

SEQ ID NO: 43 - protein, 44 aa, synthetic construct; identical to SEQ ID NO: 20
  REGION 1..44: note = FN1 domains from fibronectin

SEQ ID NO: 44 - protein, 43 aa, synthetic construct
  REGION 1..43: note = FN1 domain from t-plasminogen activator
VICRDEKTQMIYQQHQSWLRPVLRSNRVEYCWCNSGRAQCHSV

SEQ ID NO: 45 - protein, 41 aa, synthetic construct
  REGION 1..41: note = FN1 domain from HGF activator
EKCFDETRYEYLEGGDRWARVRQGHVEQCECFGGRTWCEGT

SEQ ID NO: 46 - protein, 41 aa, synthetic construct
  REGION 1..41: note = FN1 domain from Coagulation factor XII
EKCFEPQLLRFFHKNEIWYRTEQAAVARCQCKGPDAHCQRL